(19) United States
(12) Patent Application Publication
(10) Pub. No.: US 2016/0278724 A1
(43) Pub. Date: Sep. 29, 2016

(54) ROBOTIC MULTI-MODE RADIOLOGICAL SCANNING SYSTEM AND METHOD

(71) Applicant: George Papaioannou, New York, NY (US)

(72) Inventor: George Papaioannou, New York, NY (US)

(21) Appl. No.: 15/082,854

(22) Filed: Mar. 28, 2016

Related U.S. Application Data
(60) Provisional application No. 62/139,256, filed on Mar. 27, 2015.
(60) Provisional application No. 62/313,968, filed on Mar. 28, 2016.

(51) Int. Cl. A61B 6/00; G01N 23/04; A61B 6/10; G21K 1/04

(52) U.S. Cl. CPC A61B 6/4458; G21K 1/04; A61B 6/508; G01N 23/04; A61B 6/4417; A61B 6/102

(57) ABSTRACT

A robotic scanning system is provided. The scanning system includes a robotic array having at least one set of scanning robots configured to perform a radiological scan on a subject, each robot having a respective emitter or detector. A control unit in electrical communication with the robotic array controls the set of scanning robots to perform the radiological scan in accordance with scan settings received from a work station. The work station is configured to permit a user to select a radiological scan to perform on the subject from a plurality of different types of radiological scans selectable by the user. An image processing device in the system receives scan data from the robotic array and produces image data indicative of a multi-dimensional image of at least a portion of the subject.

### CROSS-REFERENCE TO RELATED APPLICATION

This application claims the benefit of U.S. Provisional Patent Application Ser. No. 62/313,968 filed on Mar. 28, 2016 and Provisional Patent Application Ser. No. 62/139,256 filed on Mar. 27, 2015, both of which are expressly incorporated herein in their entirety by reference thereto.

### FIELD OF INVENTION

Embodiments of the present invention are related to radiological scanning systems and, in particular, scanning systems and methods associated with radiology.

### BACKGROUND OF THE INVENTION

It is common in the field of radiology to conduct one or more radiological scans on various different subjects, such as patients in a hospital, animals at a veterinary clinic or in other settings, or of objects, for example, in industrial settings. However, due to limitations in current technology, radiological scanning equipment is often times configured to perform only a particular type of scan. In such a situation, a radiologist or other personnel desiring to perform multiple types of scans on a patient or subject would be required to purchase and utilize different types of radiological equipment at great expense. Multiple scans would also require moving the patient or subject from one radiological scanner to another, sometimes in different rooms of a venue, such as a hospital. Depending on the situation, the multiple scan regimen may be very time consuming, and, in the event of scans on animal or human subjects, may require anesthetizing the subject multiple times. This adds even more expense to the process and often times results in an unpleasant experience for the subject being scanned. Furthermore, since different radiological systems operate in accordance with their own protocols and coordinate systems, it may be difficult to generate composite, hybrid imagery from multiple types of scans of the subject.

### SUMMARY OF THE INVENTION

To address these and other problems in the prior art, embodiments of the present invention provide robotic radiological scanning systems configured to operate in multiple modalities to perform multiple types of radiological scans. In one embodiment, for example, a robotic scanning system is provided. The system includes a robotic array having at least one set of automated scanning robots (one with an emitter and another with a detector) configured to perform a radiological scan on a subject, such as a patient in a hospital setting or an animal, such as a horse. The system also includes a control unit in electrical communication with the robotic array. The control unit is configured to control the set of scanning robots to perform the radiological scan. A work station is also provided to transmit scan settings selected by a user and to direct the control unit to perform any of a plurality of different types of radiological scans selectable by the user. An image processing device of the system receives and processes image frames from the robotic array to produce image data indicative of a multi-dimensional image of at least a portion of the subject.

In accordance with another embodiment of the present invention, the emitter and/or detector are configured to be selectively attached to and detached from the scanning robots in accordance with a particular type of scan to be performed. This embodiment provides further flexibility that permits the robotic scanning system to adapt to perform any of a multitude of different types of radiological scans. In still another embodiment, the scanning robots are configured to automatically attached and detach themselves to a set of modular emitters and/or detectors positioned within an operational envelope of the system.

In still another embodiment of the present invention, the robotic scanning system is provided with a vision system device and a plurality of cameras positioned to view the subject and robotic array during the scan. The vision system, using the cameras, continually monitors the locations of various markers positioned on the subject and at other locations within the operational envelope of the robotic array. The vision system uses the locations of these markers to generate correction information used to compensate for offsets in image frames caused by motion of the subject with respect to the robotic scanning system. In one embodiment, the vision system generates the correction information by (i) determining a position of a first origin of a first coordinate system assigned to the subject, (ii) determining a position of a second origin of a second coordinate system assigned to the robotic array, and (iii) generating at least one correction vector in accordance with the positions of the first and second origins with respect to an origin of a fixed third coordinate system. In still another embodiment, at least some of the plurality of markers are positioned within the operational envelope in a predefined geometric pattern to assist the vision system device to distinguish between the subject and system markers.

In yet another embodiment in which the subject is a horse, a stand is provided. The stand has a base unit, an arm coupled to the base unit, and a cradle coupled to the arm and configured to receive the head of the horse during the radiological scan. With respect to a variant of this embodiment, additional markers are positioned on the stand to assist the vision system device to generate the correction information. The vision system generates this information in still another embodiment by (i) determining a position of a first origin of a first coordinate system assigned to the horse, (ii) determining a position of a second origin of a second coordinate system assigned to the robotic array, (iii) determining a position of a third origin of a third coordinate system assigned to the stand, and (iii) generating at least one correction vector in accordance with the positions of the first, second and third origins with respect to an origin of a fixed fourth coordinate system.

### BRIEF DESCRIPTION OF THE DRAWINGS

FIG. 1a is a diagram depicting components of a robotic multi-mode radiological scanning system in accordance with the present invention.

FIG. 1b is a side view of a scanning robot in accordance with the present invention.

FIG. 1c is a top view of a scanning robot side view of a scanning robot 100 in accordance with the present invention.

FIG. 1d is a side view of a scanning robot showing various physical dimensions in accordance with the present invention.

FIG. 2 is a perspective view of an emitter and a detector in accordance with the present invention.

FIG. 3 is another perspective view of an emitter and a detector in accordance with the present invention.

FIG. 4 is a perspective view of a scanning robot with modular detectors in accordance with the present invention.

FIGS. 5a through 5d show various different trajectories for emitters and detectors in accordance with the present invention.

FIG. 6 is a perspective view of a robotic array performing a panoramic scan of a subject in accordance with the present invention.

FIG. 7 is a perspective view of a robotic array performing a tomosynthesis scan of a subject in accordance with the present invention.

FIG. 8 is a perspective view of a robotic array performing a CT scan of a subject in accordance with the present invention.

FIG. 9 is a perspective view of a robotic array performing a roentgen stereophotogrammetric panoramic scan of a subject in accordance with the present invention.

FIG. 10 is a perspective view of a robotic array performing a DRSA scan of a subject in accordance with the present invention.

FIG. 11 is a perspective view of a robotic array performing a panoramic scan of an animal in accordance with the present invention.

FIGS. 12a and 12b are perspective views of a robotic array performing tomosynthesis scans of various anatomical features of an animal in accordance with the present invention.

FIG. 13 is a perspective view of a robotic array performing a 360 digital radiography scan of an animal in accordance with the present invention.

FIG. 14 is a perspective view of components of a robotic array performing a scan of an animal with inhomogeneous and variable geometry and topology emitter and detector trajectories in accordance with the present invention.

FIG. 15 is a perspective view of a robotic array performing a DRSA scan of an animal in accordance with the present invention.

FIG. 16 is a flow diagram showing steps of a process for performing a radiological scan of a subject in accordance with the present invention.

FIG. 17 is a diagram depicting components of another robotic multi-mode radiological scanning system in accordance with the present invention.

FIGS. 18a and 18b are different perspective views of a robotic array performing a radiological scan of an animal in accordance with the present invention.

FIG. 19 is a frontal view of a detector with an attached markers and cameras in accordance with the present invention.

FIG. 20 is a diagram showing vector transformation in accordance with the present invention.

FIG. 21 is another diagram showing vector transformation of an object in accordance with the present invention.

FIG. 22 is a diagram showing vector rotation in accordance with the present invention.

FIGS. 23a and 23b are diagrams showing another vector rotation in accordance with the present invention.

FIG. 24 is a diagram showing reference point coordinates in accordance with the present invention.

FIG. 25 is a diagram showing vector transformations in accordance with the present invention.

### DETAILED DESCRIPTION

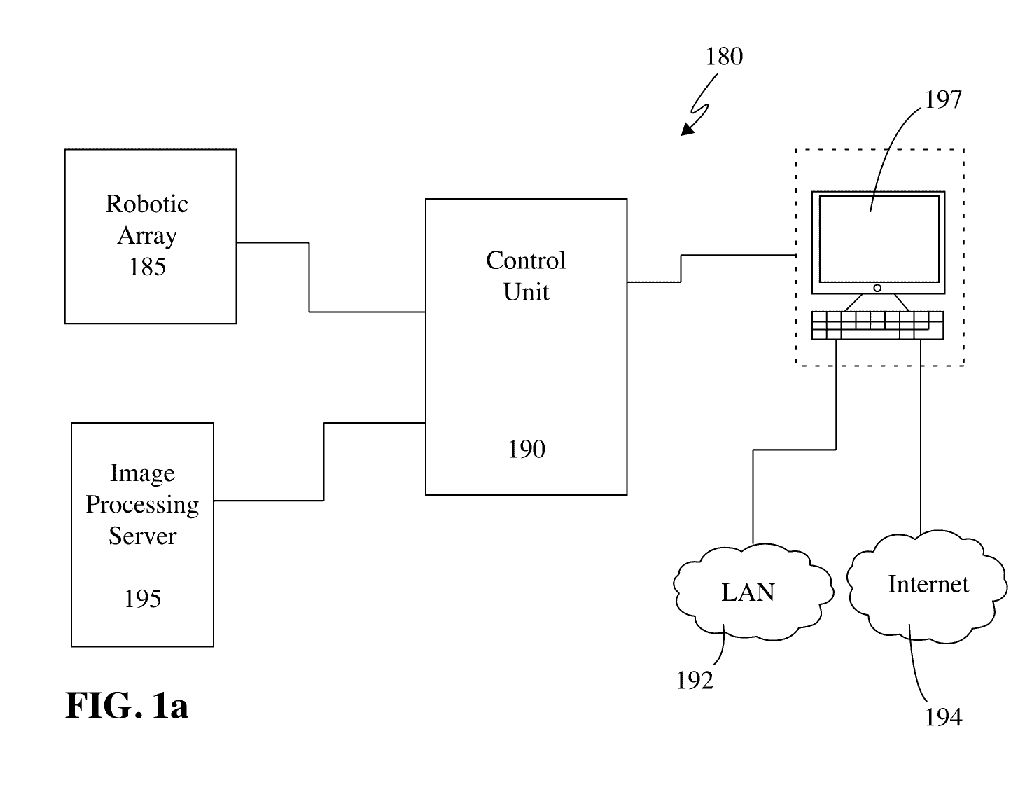

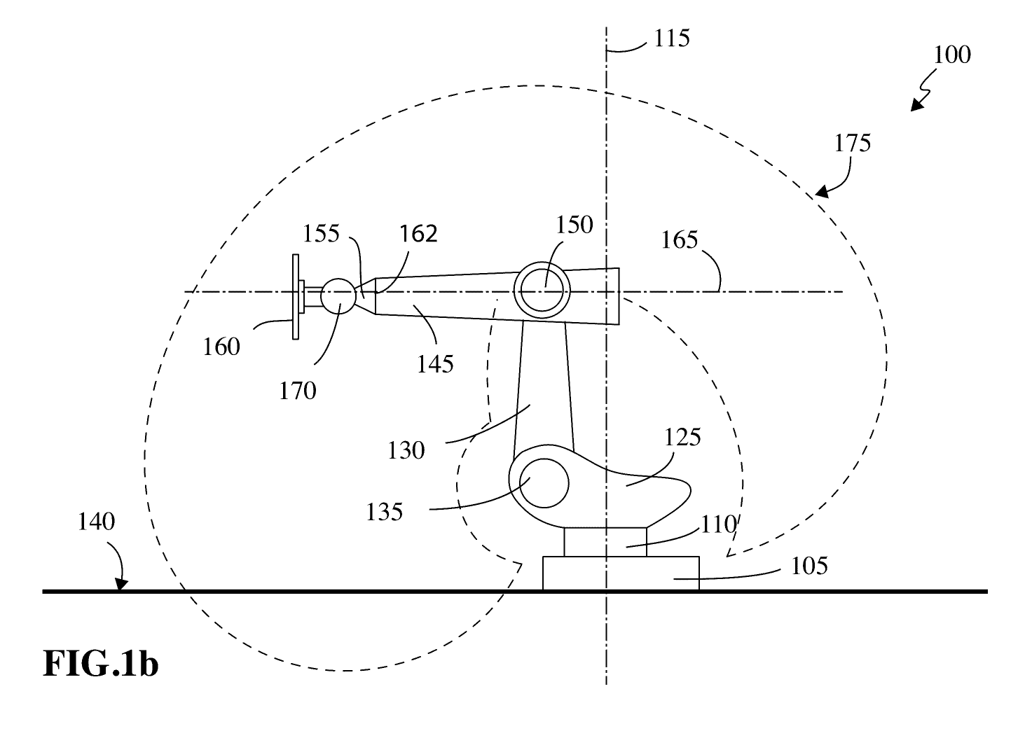

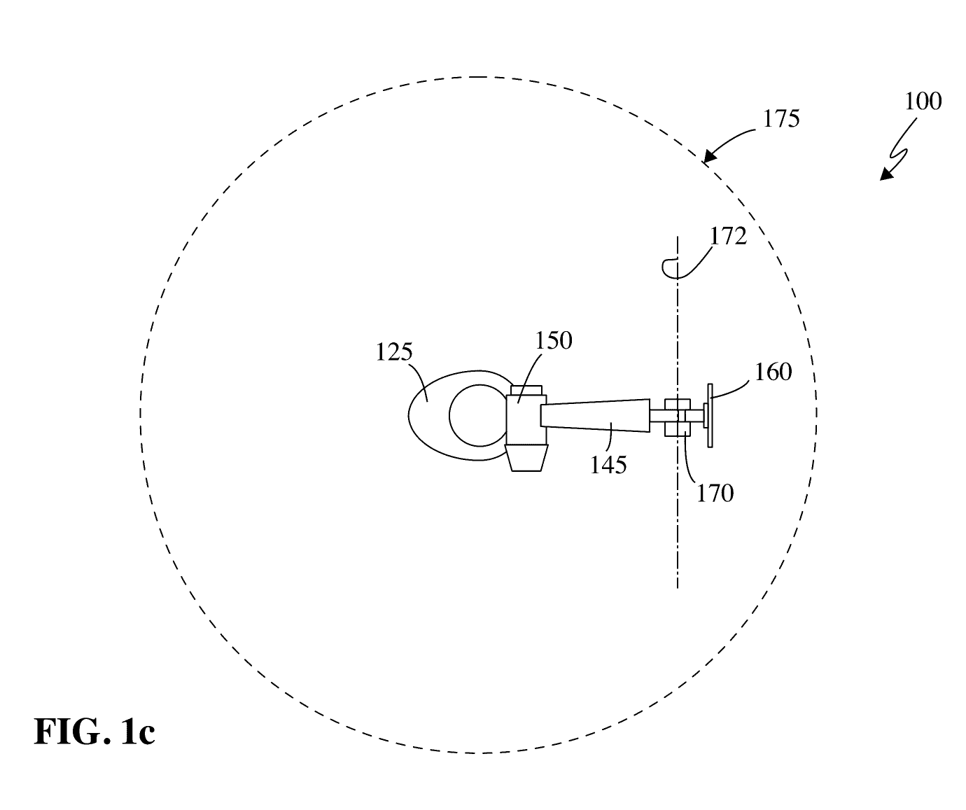

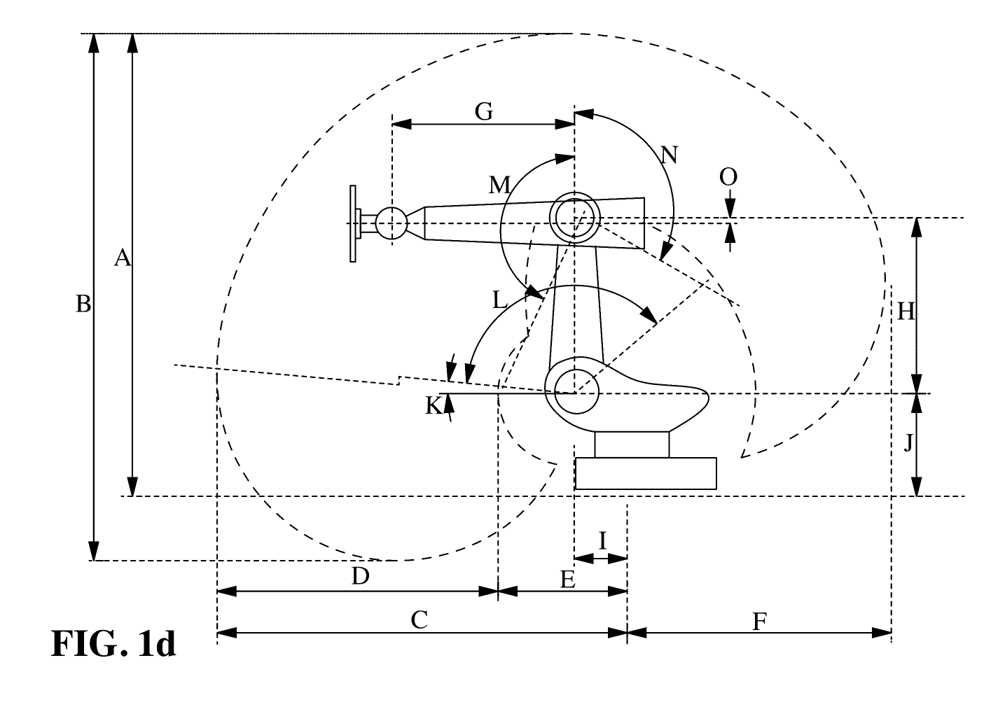

Referring now to FIG. 1a, there is seen a robotic multi-mode radiological scanning system 180 in accordance with the present invention. As more fully described below, scanning system 180 is capable of operating in multiple modalities to perform various types of radiological scans on subjects, such as persons, animals or objects. The various types of scans include, but are not limited to, panoramic scans, tomosynthesis scans, volumetric computerized axial tomography scans (volumetric CT scans), densitometry scans (or qualitative CT scans), biplane dynamic radiographic roentgen stereophotogrammetric (“DRSA”) scans, molecular (gamma) scans, and other fluoroscopy scans.

Scanning system 180 includes a robotic array 185, a control unit 190 electronically coupled to robotic array 185, an image processing server 195 electronically coupled to control unit 190, and a user work station 197 electronically coupled to control unit 190. Electronic connectivity among robotic array 185, control unit 190, image processing server 195 and user work station 197 may be effectuated using any communication medium operable to permit electronic communications, such as, for example, an intranet, a wired Ethernet network, a wireless communication network (such as Wi-Fi or Bluetooth), direct conduit wiring and/or any combination of these or other communication mediums.

Control unit 190 consists of hardware and/or software operable to control robot array 185 to perform scans in accordance with instructions received from user workstation 197. For this purpose, control unit 190 may include a general purpose computer or other off-the-shelf components executing appropriate software or, alternatively, may include special purpose hardware and/or software. In one embodiment, control unit 190 consists of one or more rack mounted personal computers (PC) operable to execute specially designed software for performing all controller functions. It will be appreciated, however, that various embodiments of the present invention are not intended to be limited to any particular processing hardware and/or software.

Image processing server 195 consists of hardware and/or software operable to process scan data acquired by robotic array 185 into image sets and other data, such as multi-dimensional images of a subject scanned by system 180. Like control unit 190, image processing server 195 may include a general purpose computer or other off-the-shelf components executing appropriate software or, alternatively, may include special purpose hardware and/or software. It will be appreciated, however, that various embodiments of the present invention are not intended to be limited to any particular image processing hardware and/or software.

User work station 197 includes hardware and/or software operable to receive commands and other instructions from radiological technicians, administrative staff or other authorized personnel for performing various functions of scanning system 180, such as selecting/customizing scanning protocols and instructing scanning system 180 to perform various types of scans. In one embodiment, user work station 197 includes a personal computer (“PC”) executing appropriate software with or without a touchscreen interface for displaying information to and receiving inputs from a user. User work station 197 may also include interface circuitry for connecting to a Local Area Network (LAN) 192 or Wide Area Network (WAN) 194, such as the Internet. Access to the LAN 192 and/or Internet permits scanning system 180 to be operated remotely, for example, from an administrative computer at a particular customer site, such as a hospital, or from one or more computers connected to the Internet.

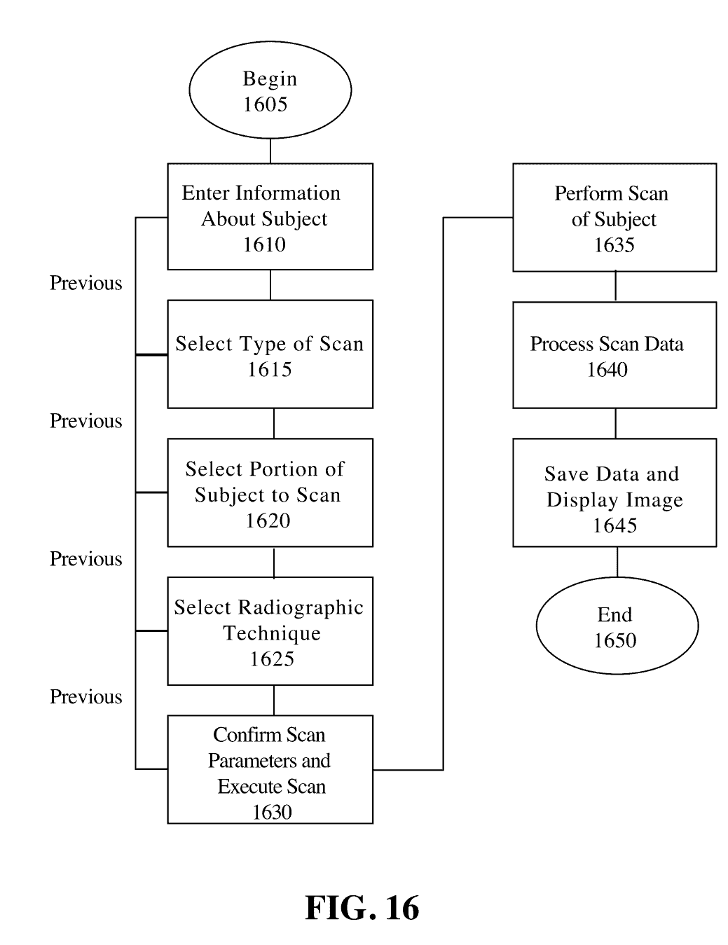

Referring now to FIG. 16 there is seen a process for using work station 197 to perform a scan in accordance with the present invention. The scan process begins at step 1605 and proceeds to step 1610, at which a user enters information about a subject to be scanned, such as a patient in a hospital or an animal (such as a horse). Information entered by the user at step 1605 may include, for example, the name of the patient or animal, the address of the patient or owner of the animal, an email address and a telephone number.

The process then proceeds to step 1615. At this step, the user selects the type of scan to perform on the subject. Types of scans may be presented to the user as a text-based list of scan types or as a series of graphical icons depicting different types of scans. In one embodiment, scan types presented to the user include graphical icons depicting a computed tomography scan, a traveling tomosynthesis (or 360 DR) scan, a roentgen stereophotogrammetric tomosynthesis scan (i.e., a biplane tomosynthesis scan using two imaging panels and two emitters), a panoramic scan, a densitometry scan, a gamma camera scan, a standard tomosynthesis scan or other radiographic scans. The user is also presented with a “previous” button that permits the user to revert back to step 1610 to correct or re-enter information about the subject to be scanned.

After the type of scan is selected, the process proceeds to step 1620, at which the user selects a portion of the subject to scan. For example, if scanning system 180 is to be used to scan a small or large animal or veterinary patient, such as a horse, the user may select from one of various different anatomical regions of the animal to scan, such as the head, neck, torso, or legs. In one embodiment, one or more regions is divided into sub-regions from which the user may select a targeted scan. For example, after selecting an option to scan the head of a horse, the user may be presented with further options allowing him/her to select a region of the head to scan. In still another embodiment, the user is requested in step 1620 to input various physical parameters associated with the subject to be scanned, such as the height of the subject (or region of the subject) off the floor. The user is also presented with a “previous” button that permits the user to revert back to step 1615 to correct or reselect the type of scan to perform on the subject.

After the user selects a portion of the subject to scan, the process proceeds to step 1625, at which the user is presented with options for selecting the radiographic technique of the scan. For example, in one embodiment, the user is presented with options for selecting either a radiographic scan technique or a fluoroscopy scan technique. The user may also be presented with options for selecting various settings associated with the selected radiographic technique. Settings may include, for example, a scanning rate in frames-per-second, source-to-object distance (“SOD”) for the subject, object-to-image receptor distance (“OID”) for the subject, scan intensity, scan power, and/or focal spot size. The user is also presented with a “previous” button that permits the user to revert back to step 1620 to correct or reselect a portion of the subject to scan.

After the user selects the radiographic technique and associated settings, the process proceeds to step 1630, at which the user is presented with the parameters of the scan selected by the user in steps 1615, 1620 and 1625. If the parameters are correct, the user may initiate the scan via an “execute scan” button. In one embodiment, at least a portion of the scan parameters is stored in memory to permit the user to recall the parameters at a later time in order to conduct a similar scan on the same or different subject. The user is also presented with a “previous” button that permits the user to revert back to step 1625 to correct or reselect a radiographic technique and associated settings.

If the user selects the “execute scan” option, the process proceeds to step 1635, at which user work station 197 instructs control unit 190 to operate robotic array 185 to perform a scan of the subject in accordance with the protocols and parameters selected by the user at steps 1615, 1620 and 1625.

After the scan is finished, the process proceeds to step 1640. At this step, data and other imagery generated by the scan are passed to control unit 190, pre-processed and forwarded to image processing server 195. Image processing server 195 then processes the data to generate various types of images, such as two-dimensional images, three-dimensional images, and/or four-dimensional (or moving three-dimensional) images. In one embodiment, image processing server 195 performs only partial processing of the imagery and other data, with at least a portion of the processing being performed by a remote server connected to LAN 192 or to the Internet.

The process then proceeds to step 1645, at which data representing these images are stored in an appropriate format (such as .pdf, .mov, .jpg format for the execution commands and imaging file formats for the scan datasets) and/or forwarded to work station 197 for display to the user. The process then ends at step 1650.

Robotic array 185 includes one or more scanning robots for performing the radiological scans initiated by user workstation 197. Referring now to FIGS. 1b and 1c, there is seen side and top views of an articulated scanning robot 100 in accordance with the present invention. Robot 100 includes a base portion 105 with a first motorized rotatable joint 110 coupled to platform unit 125. First motorized rotatable joint 110 is configured to permit controllable 360-degree rotation of robot 100 about a vertical axis 115. In one embodiment, base 105 also includes a drive mechanism (not shown) configured to permit robot 100 to be controllably moved around a circular track (not shown).

Robot 100 further includes a first arm 130 pivotally connected to platform unit 125 via a first motorized pivot 135. First motorized pivot 135 is operable to permit first arm 130 to be controllably pivoted into any of various angular positions with respect to horizontal 140, such as, for example, any angular position between 0 degrees and −140 degrees. Robot 100 also includes a second arm 145 pivotally connected to first arm 130 via a second motorized pivot 150. Second motorized pivot 150 is operable to permit second arm 145 to be controllably pivoted into any of various angular positions with respect to vertical axis 115, such as, for example, any angular position between −120 degrees and +155 degrees.

Robot 100 also includes a rotatable segment 155 coupled to second arm 145 via a second motorized rotatable joint 162. Second motorized rotatable joint 162 permits rotatable segment 155 to be controllably rotated into any angular position about second arm axis 165. Rotatable segment 155 also includes a third motorized pivot 170 connected to a radiological unit 160. Third motorized pivot 170 is operable to permit radiological unit 160 to be controllably pivoted into any of various angular positions with respect to pivot axis 172. In operation, robot 100 is operable to position radiological unit 160 in any orientation and at any point within operational envelope 175. FIG. 1d shows robot 100 with various dimensions A-O, each of which may be customized for a particular application. In this way, embodiments of the present invention provide robots 100 with scalability.

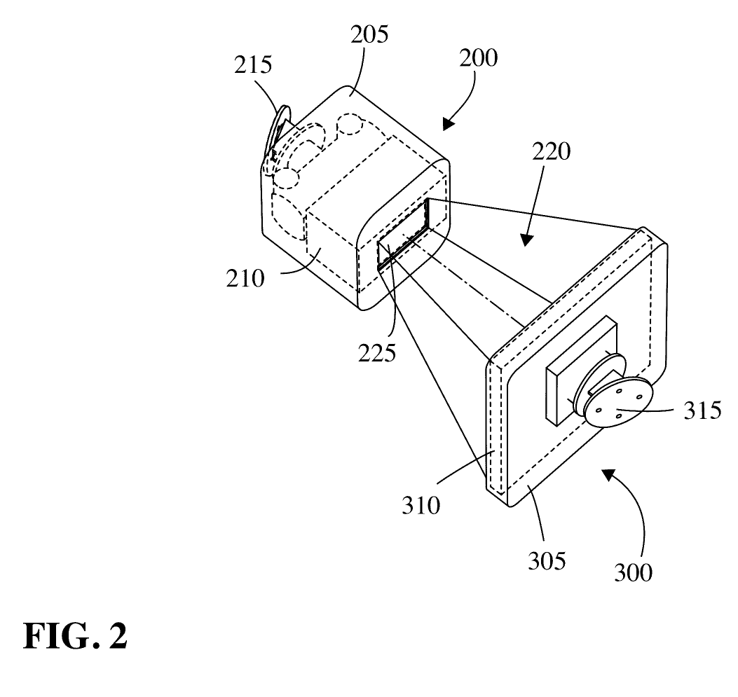

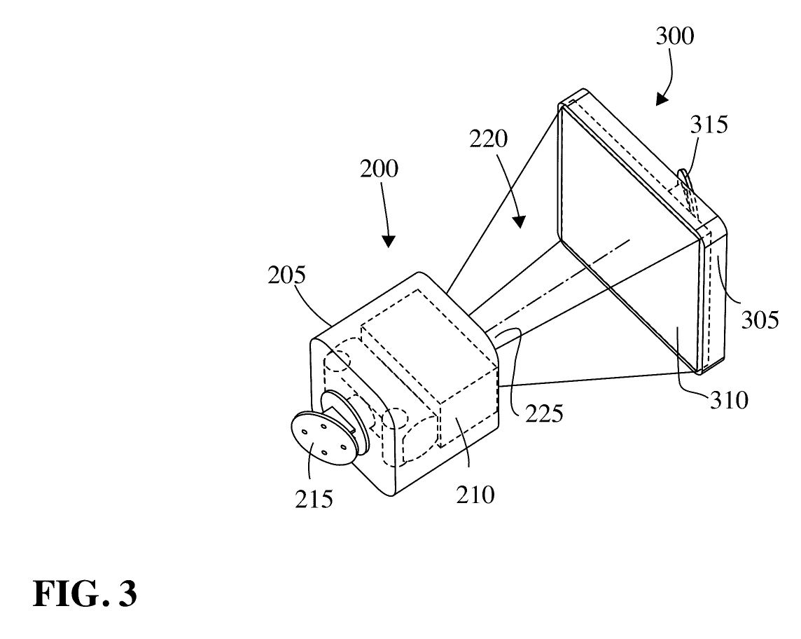

Radiological unit 160 may include any of various radiological emitters used in the field of radiology, such as, for example, emitter 200 shown in FIGS. 2 and 3. Emitter 200 includes an emitter housing 205, an emitter source 210 within housing 205, and an emitter coupling 215 for mechanically and rigidly connecting emitter 200 to rotatable segment 155 of robot 100. Emitter source 210 is operable to emit a beam 220 of one or more forms of electromagnetic radiation from wave delivery port 225, such as, for example, x-rays and/or gamma rays. In one embodiment, emitter 200 also includes a high speed shutter (not shown) positioned in front of wave delivery port 225 and synchronized with both an x-ray generating source and with a detector, such as a camera based image intensifier. The shutter operates synchronously with the x-ray generator at speeds of up to 1000 frames per second to block emission of beam 220 at times when the detector is not processing the received beam 220, such as, for example, when the shutter of a detector camera is closed. In this way, radiation dosage through a subject, such as a person or animal, may be reduced without sacrificing performance of the system.

Emitter source 210 may be of any size and have any milliamperage (MA), kilovoltage (kVp) or exposure rating. Emitter 200 may also include a collimator 230 or other device for narrowing or shaping beam 220 into any desired shape, such as a fan or cone shape, and/or for modifying the field of view of beam 220 with respect to a radiological detector used in conjunction with emitter 200. In one embodiment, emitter source 210 includes a B-150H or B-147 x-ray tube manufactured by Varian Medical Systems and an Indico 100 (80 kW) x-ray generator.

All components of emitter source 210 may be positioned entirely within emitter housing 205, as shown in FIGS. 2 and 3, or alternatively only a subset of such components may be positioned therein. In one embodiment, for example, components necessary to generate beam 220 are contained within an enclosure (not shown) separate from robot 100 and connected to delivery port 225 via a wave guide operable to guide beam 220 for emission via port 225.

Radiological unit 160 may alternatively include any of various radiological detectors used in the field of radiology, such as, for example, detector 300 shown in FIGS. 2 and 3. Detector 300 includes a detector housing 305, a detecting unit 310 within housing 305 and a detector coupling 315 for mechanically and rigidly connecting detector 300 to rotatable segment 155 of robot 100. Detector 300 is operable to receive one or more beams of electromagnetic radiation, such as beam 220, and to generate optical or electrical signals indicative of various attributes of beam 220, such as contrast and intensity at various points within beam 220. These signals are then processed and converted into images or motion capture video, usually of a subject irradiated by beam 220. Detecting unit 310 may be of any size or shape, and may include bone density detecting units or indirect detecting units, such as image intensifiers or scintillators, direct semiconductor based detecting units, such as flat-panel detecting (“FPD”) matrices, charge-coupled device (“CCD”) cameras, gamma cameras, gas-based detectors, spectrometers, silicon PN cell detectors, SPECT-CT, PET or MM compatible detectors, etc. In one embodiment, detecting unit 310 includes a PaxScan 4343CB FPD digital x-ray imaging device, designed specifically to meet the needs of Cone Beam x-ray imaging applications featuring multiple sensitivity ranges and extended dynamic range modes. In another embodiment, detecting unit 310 includes a CMOS sensor based camera operating at up to 10000 frames per second and up to a 2400×1800 native pixel resolution.

Different kinds of emitters 200 and/or detectors 300 may be better suited for particular scan applications. For example, scintillator-based detectors allow images of very high resolution to be captured whereas the use of image intensifiers allows images to be captured at a high rate, high resolution and with a relatively low x-ray dosage. For this reason, and in accordance with another embodiment of the present invention, radiological units 160 are designed as modules that can be selectively attached to rotatable segment 155 of robot 100 for particular scans, and detached and stored when not being used. For this purpose, emitter and detector couplings 215, 315 may be designed in such a way so as to permit emitters 200 and detectors 300 to be removably attached to rotatable segment 155. Removable attachment of emitter and detector couplings 215, 315 may be effectuated manually (such as by screws, bolts, latches or other similar means) or automatically via an electronically controllable coupling device controllable to selectively engage or disengage emitter and detector couplings 215, 315 with or from rotatable segment 155. In another embodiment, coupling 215, 315 of a specific type of emitter 200 or detector 300 may be designed to mate with a specially designed intermediate coupling device (not shown) which, in turn, couples to rotatable segment 155 of robot 100. The intermediate coupling device may be designed with additional features or functionality tailored to a specific emitter 200 or detector 300. For example, the intermediate coupling device may include a telescoping portion allowing emitter 200 or detector 300 to be controllably extended in a particular direction with respect to robot 100. The intermediate coupling device may also include additional controllable pivots and rotatable components capable of enhancing the range of motion of emitter 200 or detector 300 within operational envelope 175. In still another embodiment, the intermediate coupling device may be provided with settable joints configured to selectively change one or more angles of emitter 200 or detector 300 with respect to robot 100.

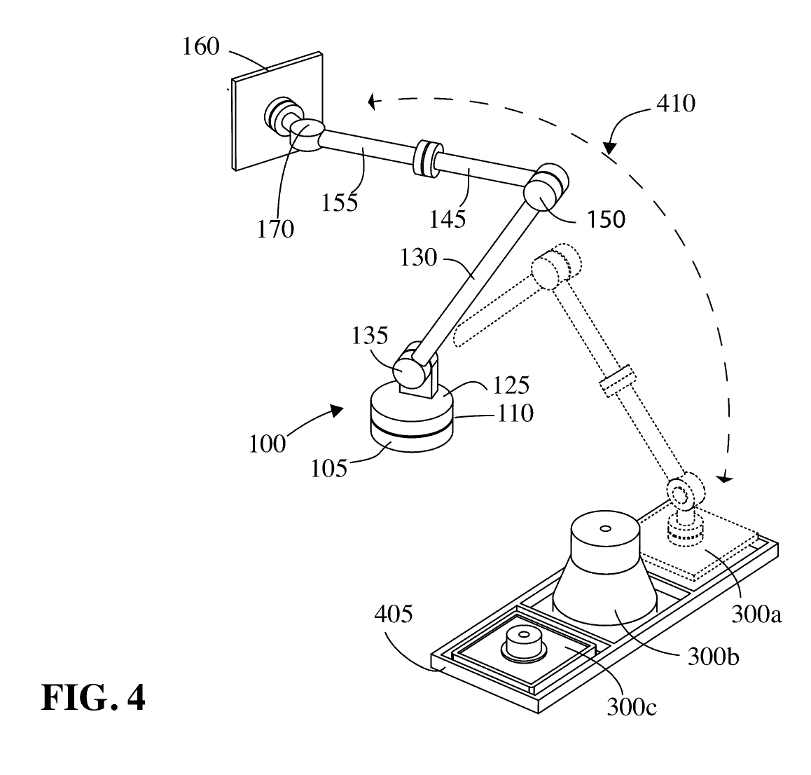

In yet another embodiment, robot 100 is operable to select and automatically attach itself to one of multiple modular emitters 200 and/or detectors 300 in accordance with a type of scan to be performed. Referring now to FIG. 4, there is seen a scanning robot 100 configured to selectively attach itself to any of three different kinds of detectors 300a, 300b, 300c in a storage unit 405 within operational envelope 175. As shown in FIG. 4, robot 100 is first controlled to position rotatable segment 155 opposite the back of a desired one of detectors 300a, 300b, 300c (in the case of FIG. 4, detector 300a). Once so positioned, rotatable segment 155 is electronically controlled to engage with detector coupling 315a of detector 300a, thereby attaching detector 300a to robot 100 (shown in dotted lines). Robot 100 then removes detector 300a from storage unit 405 along trajectory 410 to thereafter perform the desired scan. After the scan is complete, robot 100 may position detector 300a back in storage unit 405 by reversing the process described above.

It should be appreciated that, although FIG. 4 shows a robot 100 configured to selectively attach itself to one of only three detectors 300a, 300b, 300c, any number and kind of detectors may be employed. It should also be appreciated that robot 100 may be configured to selectively attach itself to any number and kind of available emitters 200 as well.

In accordance with various embodiments of the present invention, one or more sets of scanning robots 100 (one with an attached emitter 200 and another with an attached detector 300) are used together in robotic array 185 to perform one or more types of radiological scans on a subject positioned between them, such as a person, animal or object. Each set of scanning robots may be controlled to perform a stationary scan, during which emitter 200 and detector 300 remain stationary, or a moving scan, during which emitter 200 and detector 300 travel along predefined trajectories during the scan. In either case, and in accordance with one embodiment, each set of scanning robots 100 is controlled such that (i) beam 220 emitted from emitter 200 passes through an area of interest in the subject and (ii) emitter 200 and detector 300 of each set are oriented to face each other at all times during the scan. In this way, it can be better ensured that successive images captured by detector 300 during the scan are continuous and spatially aligned with respect to one another, thereby allowing the successive images and other data obtained by detector 300 to be used to construct multi-dimensional views of the area of interest. It will be appreciated by those having ordinary skill in the art that the area of interest may be a single location within the subject or, alternatively, may change over time during the scan. For example, the area of interest may follow a preset and continuous (or discrete) trajectory through the subject during the scan.

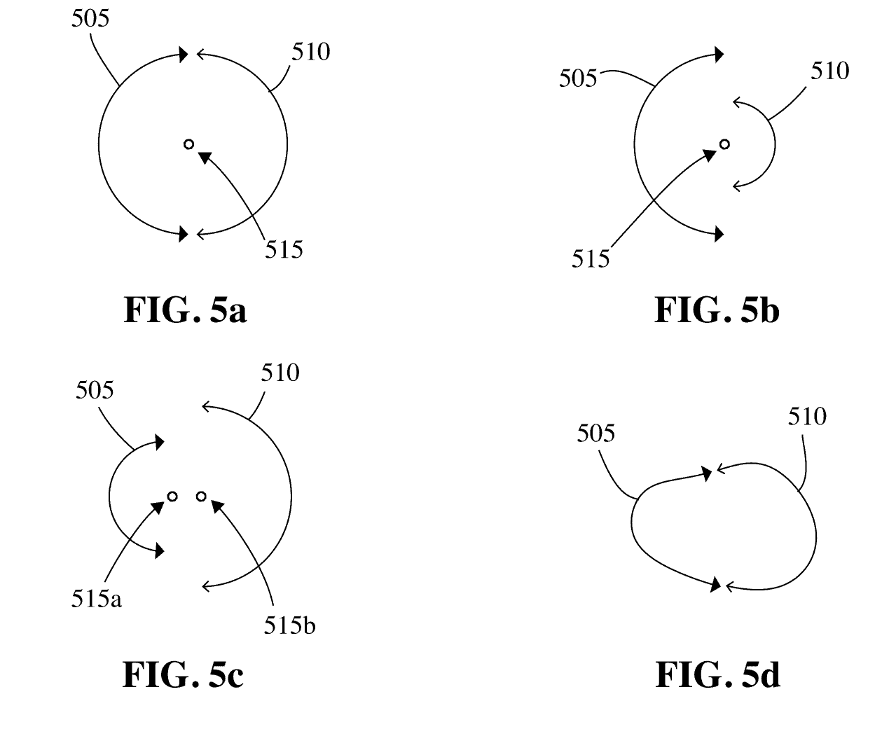

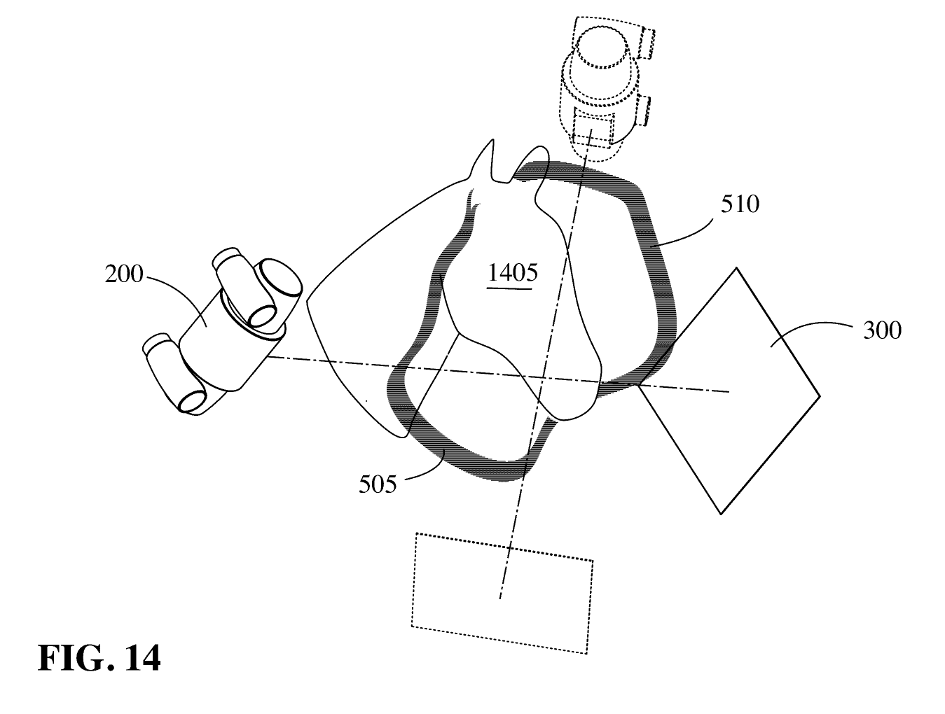

Depending on the type of scan, desired magnification of an image series, and/or other parameters, emitter 200 and detector 300 may be controlled to traverse any desired trajectories within operational envelope 175 during a scan, such as, for example, a series of points on a plane, such as a horizontal or vertical plane, or points along any path in three dimensions within operational envelope 175. Referring now to FIGS. 5a through 5d, there are seen various different scan trajectories 505, 510 of emitter 200 and detector 300, respectively. FIG. 5a, for example, shows concentric scan trajectories 505, 510 of emitter 200 and detector 300 each with the same diameter. With respect to such a scan, the area of interest of the subject is stationary and located at a focal point 515 of concentric scan trajectories 505, 510, with each of emitter 200 and detector 300 being equidistant from the area of interest and from one another at all times during the scan. FIG. 5b also shows concentric scan trajectories 505, 510 of emitter 200 and detector 300, except that concentric scan trajectory 510 of detector 300 has a diameter smaller than that of scan trajectory 505 of emitter 200. Reducing the diameter of detector trajectory 510 (or OID) may be desirable to improve image contrast or increase magnification of an image series captured by detector 300. In another embodiment, such as the one depicted in FIG. 5c, scan trajectories 505, 510 are circular in shape, but are non-concentric, with each trajectory having a separate focal point 515a, 515b, respectively. Scan trajectories 505, 510 may also follow inhomogeneous pathways, such as those depicted in FIG. 5d. In such a scan the OID between the area of interest and detector 300, the SOD between emitter 200 and the area of interest, and/or the source-to-image receptor distance (“SID”) between emitter 200 and detector 300 may vary during the scan. For instance, FIG. 14 depicts a scan of a head 1405 of a horse having inhomogeneous trajectories 505, 510 for both emitter 200 and detector 300. Inhomogeneous trajectories 505, 510 and other types of trajectories, such as parallel trajectories, also permit the area of interest in the subject to change over time during the scan.

Since robots 100 may be selectively attached to different kinds of modular emitters 200 and detectors 300 and may traverse emitter 200 and detector 300 along any trajectory within operational envelope 175, robots 100 may operate in different modalities to perform various different kinds of scans, each of which is traditionally performed in the prior art by a separate radiological device.

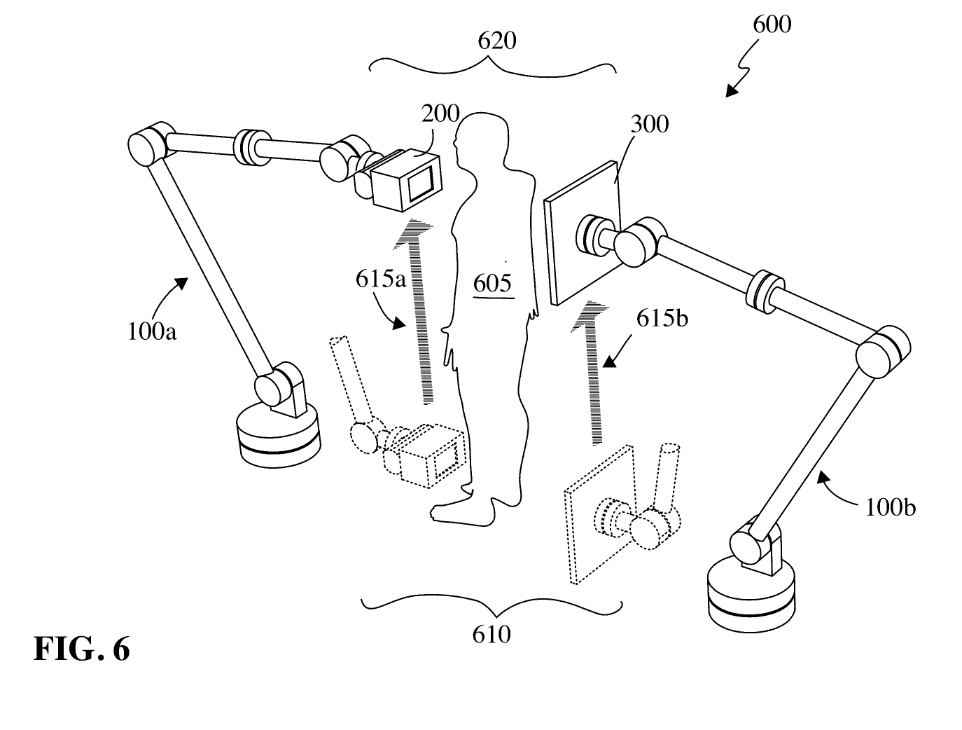

For example, referring now to FIG. 6, there is seen a robotic array 600 operating in a first modality to perform a panoramic scan of a subject 605, in accordance with the present invention. Robotic array 600 includes two scanning robots 100a (with attached emitter 200) and 100b (with attached detector 300) positioned opposite one another on either side of subject 605. The panoramic scan begins with robots 100a, 100b positioning emitter 200 and detector 300 opposite one another in a starting position 610 (shown in dotted lines). Once positioned at starting position 610, emitter source 210 of emitter 200 is activated, causing beam 220 to irradiate subject 605 and strike detector 300. While emitter source 210 remains activated, robots 100a, 100b move emitter 200 and detector 300 upward along vertical paths 615a, 615b to an ending position 620. Successive image frames obtained by detector 300 are then processed to produce a panoramic, planar image of subject 605. In another embodiment, emitter 200 and detector 300 remain stationary, while subject 605 is moved along a straight trajectory between emitter 200 and detector 300. In still another embodiment, the panoramic scan is performed using an FPD detector 300 better suited to capturing images of a subject 605 that moves with respect to detector 200 and emitter 300 during the scan, such as during a panoramic scan.

It should be appreciated that, although FIG. 6 depicts a panoramic scan along a vertical direction, the panoramic scan may be performed in any direction, such as horizontally, diagonally, or along any trajectory within operational envelope 175. For example, FIG. 11 depicts a horizontal panoramic scan of an animal, such as horse 1105. In this embodiment, the panoramic scan provides a whole-body image of horse 1105, primarily to assess angulation and the relationship of various anatomical and mechanical axes, as well as the geometric relationship of loaded structures. It should also be appreciated that robotic array 600 may include any number of scanning robots, even though only 2 scanning robots 100a, 100b are depicted in FIG. 6.

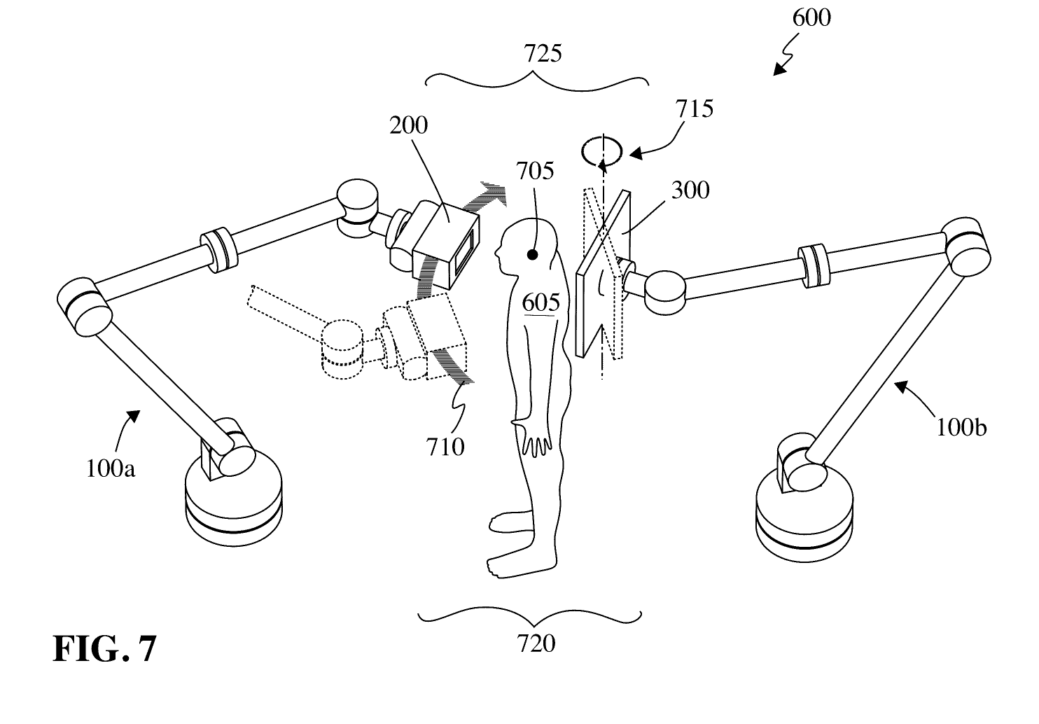

Referring now to FIG. 7, there is seen robotic array 600 operating in a second modality to perform a tomosynthesis scan of an area of interest 705 of subject 605. Tomosynthesis scans are best suited in situations where high resolution and high contrast images of area of interest 705 are desired, such as, for example, high resolution images of morphological structures of a body or animal part. Tomosynthesis provides accurate 3D static morphologic data, with ultra-thin slices (out of plane resolution) to reduce the potential of interpretation error.

As shown in FIG. 7, robot 100a performs the tomosynthesis scan by traversing emitter 200 along circular trajectory 710 from a start position 720 (noted in dotted lines) to an end position 725, such that the field-of-view of beam 220 emitted from emitter 200 is focused on area of interest 705 at all times during the scan. Robot 100b also traverses detector 300 along trajectory 715 to ensure that detector 300 faces emitter 200 during the scan. Unlike in prior art devices, the use of highly precise and accurate robots 100a, 100b in array 600 permits detector 300 to follow a trajectory 715 with an extremely small OID, thereby improving contrast and magnification in the resulting image. Detector 300 captures successive image frames of area of interest 705 during the scan, which frames are then processed to produce a high resolution, three-dimensional image of area of interest 705. In one embodiment, the tomosynthesis scan is performed using a high resolution detector 300, such as a selenium FPD detector 300, to ensure the highest resolution possible.

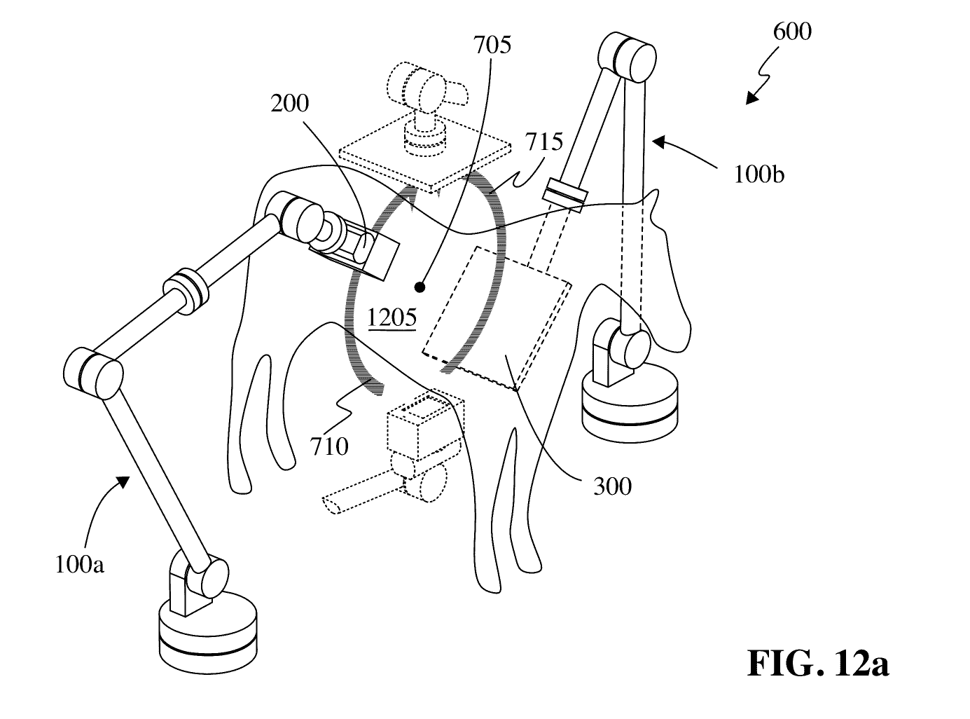

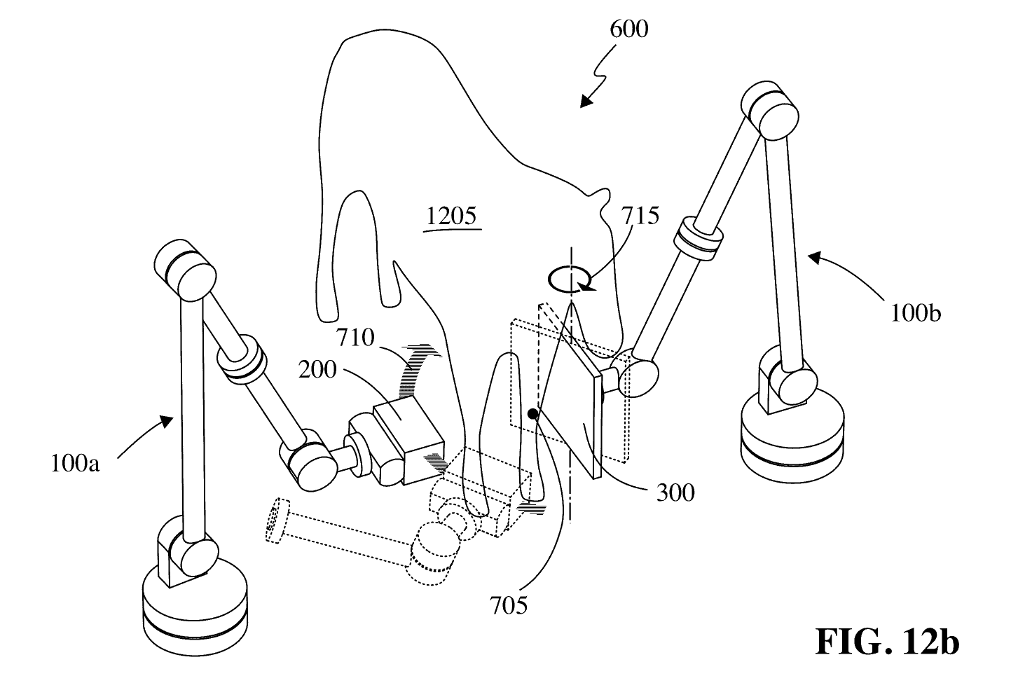

In another embodiment, such as the one depicted in FIG. 12a, robotic array 600 operates to perform a tomosynthesis scan of an animal part, such as the torso of horse 1205. Robotic array 600 may also perform a tomosynthesis scan of another anatomical part of an animal, such as a leg of horse 1205 as depicted in FIG. 12b. It should be appreciated that the configuration of robotic array 600, as shown in FIGS. 7 and 12, may be used to perform a volumetric CT scan as well.

Unlike a traditional CT scan, a tomosynthesis scan does not require emitter 200 to perform a complete 360-degree rotation around area of interest 705. Tomosynthesis scans of less than 360 degrees still produce high quality three dimensional images, albeit with a limited depth of field. Tomosynthesis scans also typically require fewer slice images for reconstruction of the three three-dimensional image, thereby reducing both cost and radiation exposure compared to traditional CT scans.

Robotic array 600 may also be operated in a third modality to perform a traveling tomosynthesis scan (or a digital radiography or 360 DR scan) of subject 605. In this embodiment, a series of tomosynthesis scans are performed such that that the focus of beam 220 emitted from emitter 200 (and thus area of interest 705) is changed slightly from scan to scan along a predefined trajectory through subject 605. Detector 300 captures successive image frames of area of interest 705 during the scan, which frames are then processed to produce a high resolution, three-dimensional image of subject 605, with increased focus and higher resolution of structures situated within the trajectory of area of interest 705 through subject 605. A traveling tomosynthesis scan is useful if high resolution images of structures larger than the field-of-view focus are desired, or for navigational guidance where high in-plane-resolution is critical to eliminate errors in surgical procedures. For example, a traveling tomosynthesis scan may be employed to produce a high resolution image of a femur bone by ensuring that the focus of successive tomosynthesis scans traverses along the central axis of the femur.

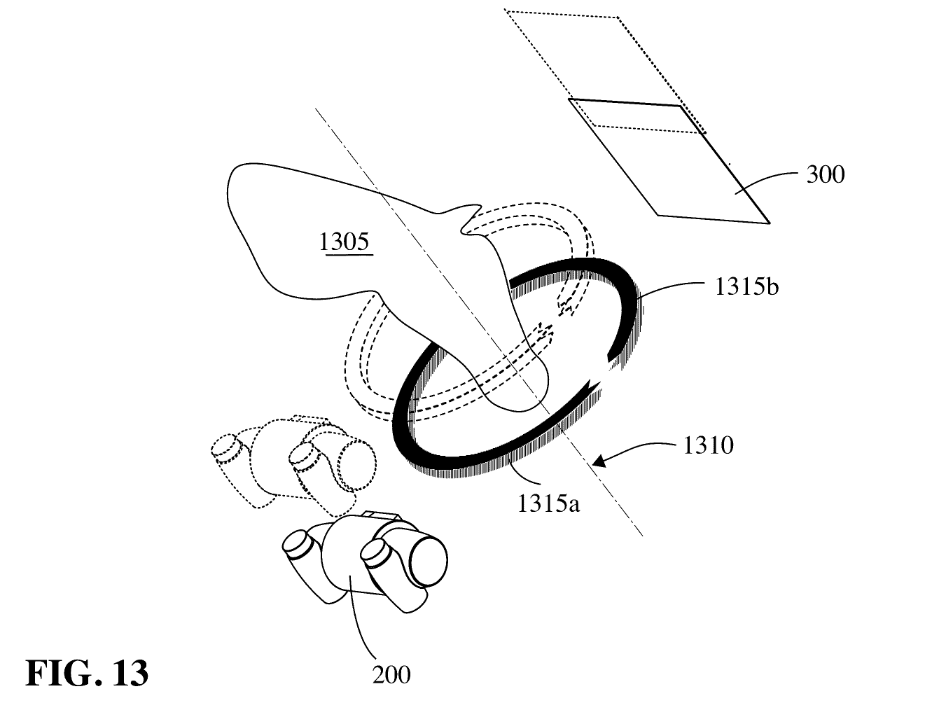

A traveling tomosynthesis scan (or 360 DR scan) may also be employed to produce a high resolution image of any anatomical feature of an animal, such as the head 1305 of a horse, such as the one depicted in FIG. 13. A shown in FIG. 13, system 600 performs a series of tomosynthesis scans of head 1305 along axis 1310. Emitter 200 and detector 300 follow respective circular trajectories 1315a, 1315b for each successive scan, with the focal point moving slightly from scan to scan along a trajectory roughly coincident with axis 1310. Successive synchronized tomosynthesis images obtained by detector 300 (or multiple detectors 300) are co-registered by image processing server 195 and combined to produce a single, high-resolution three-dimensional image of head 1305.

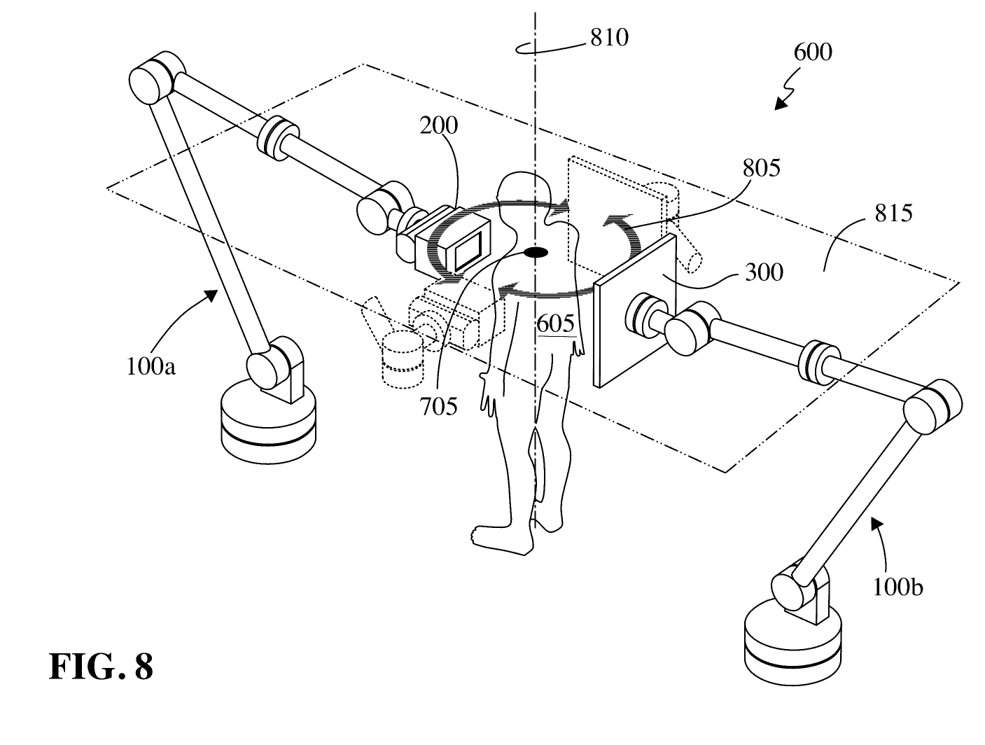

Referring now to FIG. 8, there is seen robotic array 600 operating in a fourth modality to perform a volumetric CT scan of subject 605. As shown in FIG. 8, the volumetric CT scan is performed by controlling robots 100a, 100b to traverse emitter 200 and detector 300 in opposite directions along the same circular circumference 805 about vertical axis 810. In the event that emitter 200 includes a cone-based emitter source 210 (i.e., an emitter source 210 producing beam 220 in the shape of a cone), a single 180-degree scan within plane 815 may produce an image series sufficient for reconstructing a three-dimensional representation of an area of interest 705 in subject 605. Alternatively, in the event that emitter 200 includes a fan-based emitter source 210 (i.e., an emitter source 210 producing beam 220 in the shape of a fan), multiple scans may be required to reproduce the desired three-dimensional representation. In such a situation, successive scans are offset by a small distance in a direction approximately perpendicular to plane 815. The successive scans produce co-registered image “slices” from which the three-dimensional representation of area of interest 705 may be constructed. In an alternative embodiment, emitter 200 and detector 300 are kept stationary during the scan, while subject 605 is rotated about vertical axis 810, for example, via a rotating platform (not shown). In this embodiment, and in the event that a fan-based emitter 200 is employed, emitter 200 and detector 300 are moved upwardly by a small distance after each successive rotation of subject 605 to produce the successive “slices,” from which the three-dimensional representation of area of interest 705 may be constructed.

In still another embodiment, detector 300 is replaced with a bone density flat panel detector 300 for operating system 600 in a fifth modality to perform a densitometry scan for measuring bone density. Operation of robotic array 600 to perform a densitometry scan is similar to that required for a volumetric CT scan, except that rotation of robots 100 and/or subject 605 occurs at a slower rate. In this embodiment, emitter 200 produces a series of low and high intensity beams 220 which irradiate area of interest 705. Differences in density, for example, in a bone, affect absorption of the beams 220 as they pass through subject 605, thereby producing intensity and contrast variations at detector 300. These variations are then processed by image processing server 195 to produce an image showing regions of high and low density within area of interest 705.

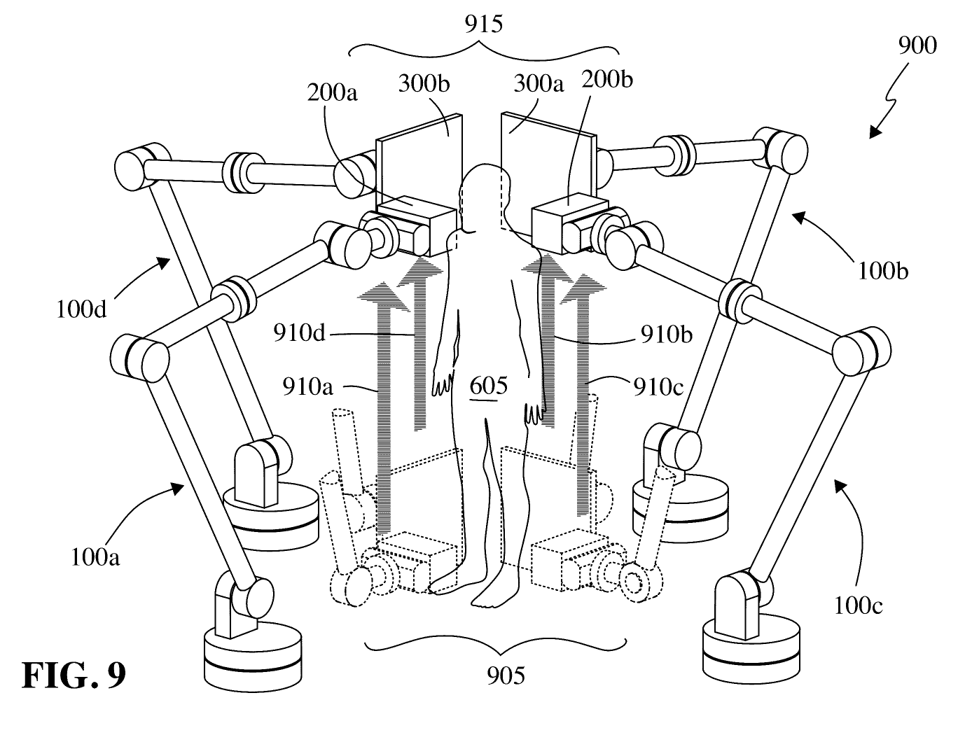

Referring now to FIG. 9, there is seen a robotic array 900 operating in a sixth modality to perform a roentgen stereophotogrammetric panoramic scan (stereo panoramic scan) of a subject 605, in accordance with the present invention. Robotic array 900 includes a first set of scanning robots 100a (with attached emitter 200a) and 100b (with attached detector 300a) and a second set of scanning robots 100c (with attached emitter 200b) and 100d (with attached detector 300b). Although only four robots 100a, 100b, 100c, 100d are shown in FIG. 9, it should be appreciated that robotic array 900 may include any number of sets of robots 100, to perform any kind of scan.

Robots 100a, 100b, 100c, 100d are positioned around subject 605 such that emitters 200a, 200b are at right angles to one another. The stereo panoramic scan begins with robots 100a, 100b, 100c, 100d positioning emitters 200a, 200b and detectors 300a, 300b opposite one another in a starting position 905 (shown in dotted lines). Once positioned at starting position 905, emitter sources 210a, 210b of emitters 200a, 200b are activated. While emitter sources 210a, 210b remain activated, robots 100a, 100b, 100c, 100d move emitters 200a, 200b and detectors 300a, 300b upwardly along vertical paths 910a, 910b, 910c, 910d to an ending position 915. Successive image frames obtained by detectors 300a, 300b are then processed to produce a co-registered, biplane image of subject 605. In another embodiment, emitters 200a, 200b and detectors 300a, 300b remain stationary, while subject 605 is moved along a straight trajectory between emitters 200a, 200b and detectors 300a, 300b. In still another embodiment, the stereo panoramic scan is performed using FPD detectors 300a, 300b. It should also be appreciated that, although FIG. 9 shows emitters 200a, 200b positioned at right angles with respect to one another, emitters 200a, 200b may be positioned at other angles. For example, in one embodiment, the stereo panoramic scan is performed using emitters 200a, 200b positioned at an angle less than 90 degrees with respect to one another.

The biplane image of subject 605 may also be processed in accordance with known roentgen stereophotogrammetric principles to produce a three-dimensional representation of at least a portion of subject 605. It can also be combined with back projection and other three dimensional reconstruction techniques for improved resolution. Stereophotogrammetry generally uses triangulation to construct a three-dimensional representation from two or more two-dimensional images (in this case, two-dimensional image sets captured by detectors 300a, 300b). Methods for processing biplane roentgen stereophotogrammetric images, including methods to correct for undesirable motion of subject 605 during the scan, are described in commonly owned U.S. Pat. Nos. 8,147,139; 8,525,833; and 8,770,838, the entire contents of which are expressly incorporated herein by reference.

In another embodiment, robotic array 900 is operable to perform other types of roentgen stereophotogrammetric scans, such as roentgen stereophotogrammetric tomosynthesis, CT, and densitometry scans. Like the roentgen stereophotogrammetric panoramic scan depicted in FIG. 9, roentgen stereophotogrammetric tomosynthesis, CT and densitometry scans employ two or more sets of robots 100a, 100b, 100c, 100d, each performing a scan in a different trajectory to acquire offset data sets which can be processed in accordance with roentgen stereophotogrammetric principles to produce and/or enhance three dimensional imagery generated from image sets.

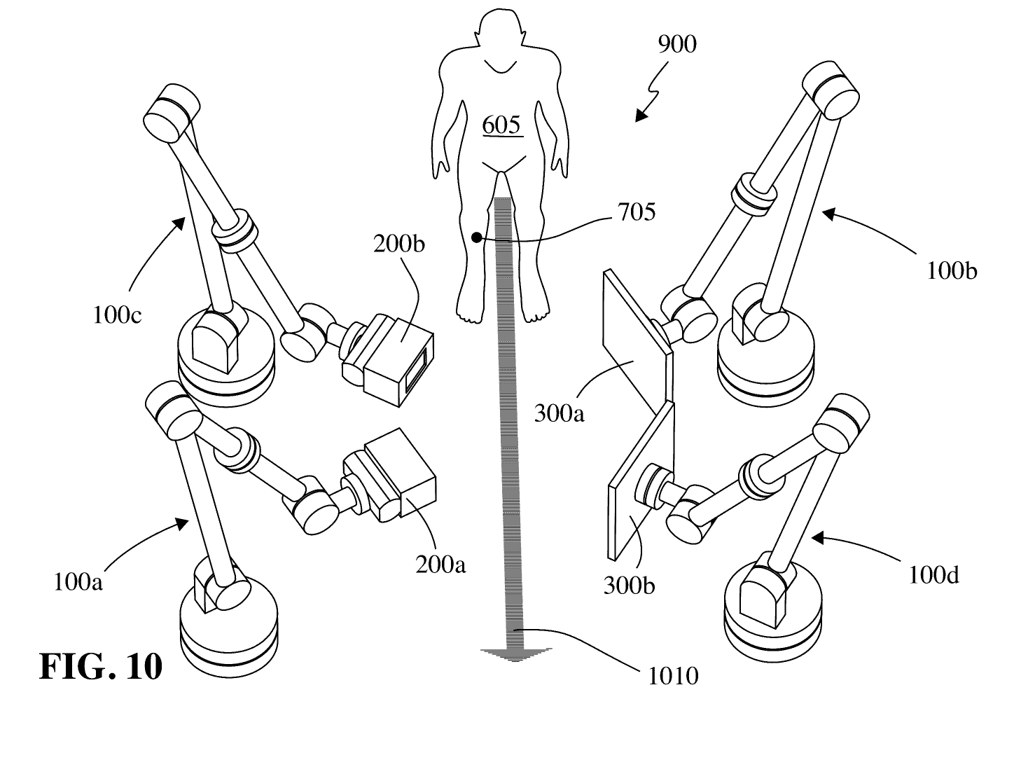

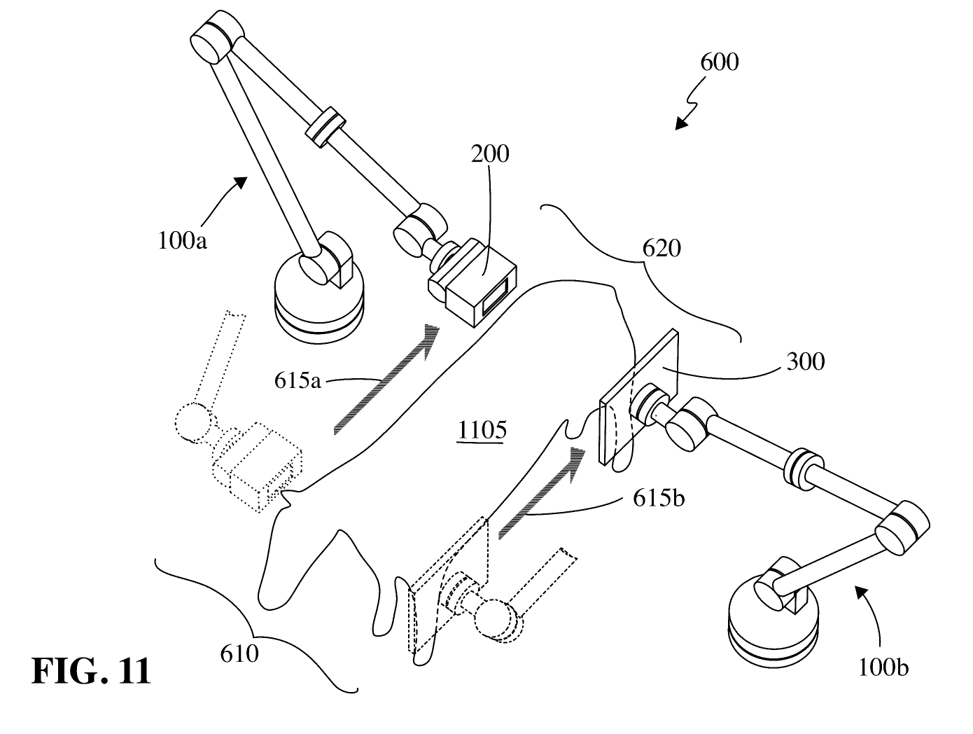

Referring now to FIG. 10, there is seen robotic array 900 operating in a seventh modality to perform a DRSA scan of an area of interest 705 in subject 605. A DRSA scan employs dynamic Roentgen stereovideoradiography techniques to provide an accurate analysis of area of interest 705 under load and in motion, such as, for example, a knee joint of a person or animal walking, jumping or running.

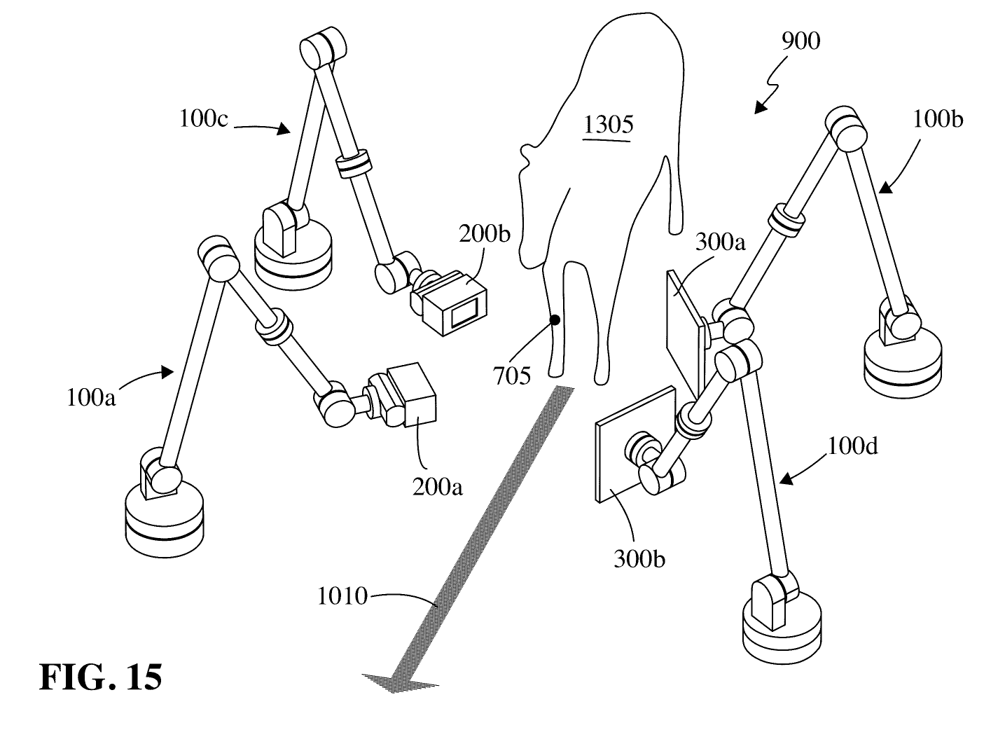

The DRSA scan begins with emitters 200a, 200b and detectors 300a, 300b positioned opposite one another within a scanning plane passing generally through area of interest 705 of subject 605 to be scanned (in one embodiment, cone-based emitters 200a, 200b are employed). After emitter sources 210a, 210b are activated, subject 605 is passed between emitters 200a, 200b and detectors 300a, 300b along a predetermined trajectory 1010. Successive image frames obtained by detectors 300a, 300b are processed in accordance with roentgen stereophotogrammetric principles (such as those described in commonly owned U.S. Pat. Nos. 8,147,139; 8,525,833; and 8,770,838, including methods to correct for undesirable motion of subject 605 during the scan) to construct a four-dimensional representation (i.e., a three-dimensional video) showing area of interest 705 in motion and under load. It should be appreciated by those having ordinary skill in the art that subject 605 need not move along trajectory 1010 (for example, when subject 605 is a person running on a treadmill during the scan). It should also be appreciated that system 900 may perform a DRSA scan of other types of motion relating to subject 605. For example, in the event subject 605 is a person, a DRSA scan may capture a four dimensional image in relation to time (i.e., a video) of an arm joint in motion or the morphological features of a person swallowing food. A DRSA scan may also show, for example, compression of cartilage at the intersection of a femur and a knee joint, as the joint absorbs load from the musculoskeletal system and is compressed during high-speed impact and support phases of running. In another embodiment, such as the one depicted in FIG. 15, robotic array 900 operates to perform a DRSA scan of an animal, such as horse 1305.

As described above, radiological scanning system 180 is capable of performing various different scans of a subject 605. Since the same robotic array 185, 600, 900 is used for each type of scan, all scans are conducted with respect to the same fixed coordinate system, for example, an x-y-z Cartesian coordinate system defining each point within operational envelope 175 of system 180 by an x, y, z coordinate. As such, all scans of the same subject 605 are automatically co-registered with one another. This permits image processing server 195 to construct hybrid images using image sets from different scans. For example, scanning system 180 may perform tomosynthesis and bone density scans of a leg bone and then produce a hybrid image showing a high resolution tomosynthesis image of the bone with bone density color gradients overlaid thereon. Or, for example, system 180 may perform roentgen stereophotogrammetric panoramic and traveling tomosynthesis scans of a person's torso. Such a scan set permits image processing server 195 to generate a composite three-dimensional image of the torso containing both the three-dimensional imagery captured by the panoramic scan and the higher resolution information of select areas of interest captured during the tomosynthesis scan. It will be appreciated by those having ordinary skill in the art that system 180 may perform any combination of scans to produce any of various different composite and hybrid images.

In some instances, undesirable motion of subject 605 during a scan (or between scans when producing hybrid images of the same subject 605) results in an offset of one or more frames in an image set captured by detector 300. The offset, if not compensated for by image processing server 195, may cause artifacts or blurriness in the final multi-dimensional image generated from the image set of frames. To adequately address these issues, it is desirable to know the position of subject 605 with respect to robotic array 600 at all times during a scan. Knowledge of the position of subject 605 at all times allows processing server 195 to compensate for any offsets in individual frames by realigning the frames (e.g., by subtracting offsets from the frames) before generating the resulting multi-dimensional image of subject 605. Methods to correct for offsets in frames of a captured image set during processing are described in commonly owned U.S. Pat. Nos. 8,147,139; 8,525,833; and 8,770,838. These methods employ artificial or natural markers within subject 605 to manually or automatically align each frame during image processing.

Embodiments of the present invention provide other methods for determining the position of subject 605 during a scan, thereby enhancing image processing and the resultant images produced by a radiological scanning system. These methods may be used on their own, or in conjunction with other methods for correcting frame offsets.

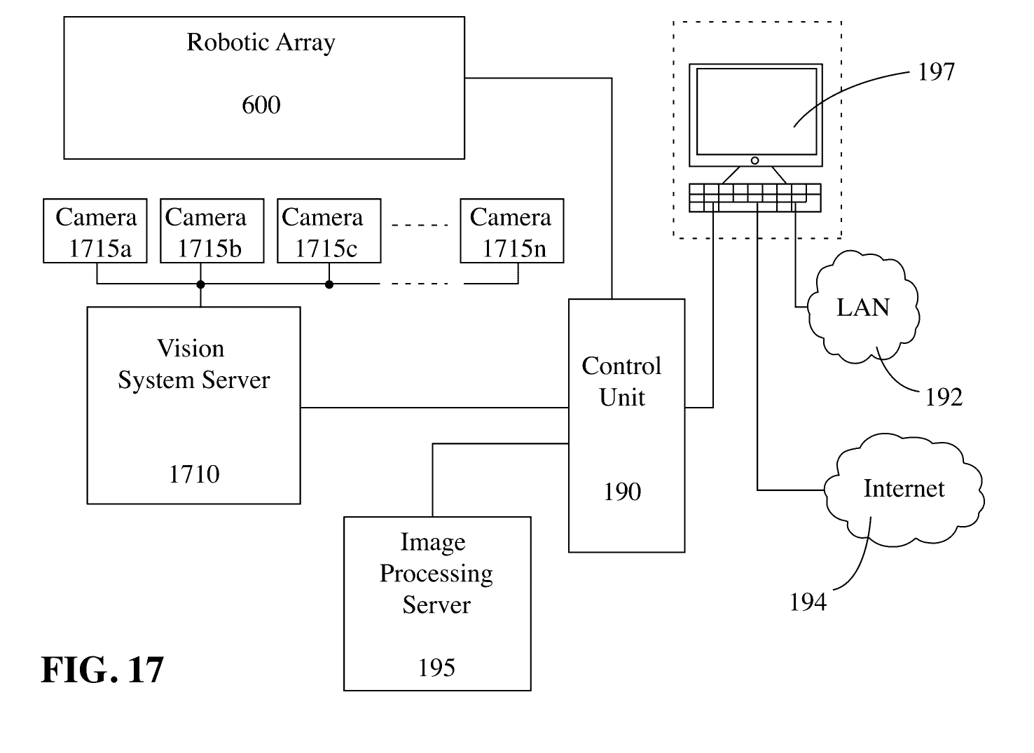

Referring now to FIG. 17, there is seen a radiological scanning system 1705 with offset correction capabilities, in accordance with the present invention. Similar to system 180, scanning system 1705 includes a robotic array 600 (or robotic array 900 in other embodiments), a control unit 190 electronically coupled to robotic array 600, an image processing server 195 electronically coupled to control unit 190, and a user work station 197 electronically coupled to control unit 190. Scanning system 1705 also includes a vision system server 1710 coupled to control unit 190 and to two or more cameras 1715a, 1715b, 1715c, . . . 1715n positioned to view robotic array 600. Some of cameras 1715a, 1715b, 1715c, . . . 1715n may be positioned at various stationary locations around robotic array 600, while others may be positioned, for example, on features of robotic array 600 itself, such as on various arms of robots 100a, 100b. Having multiple cameras 1715a, 1715b, 1715c, . . . 1715n in various positions better ensures that subject 605 may be viewed by vision system server 1710 at all times, even when various obstructions block the view of subject 605 from one or more of cameras 1715a, 1715b, 1715c, . . . 1715n.

In accordance with this embodiment, artificial markers, such as tantalum (or other) markers, are positioned both on the surface of subject 605 (subject markers) and at other known locations within robotic array 600 (system markers), such as on robots 100a, 100b themselves, on emitter 200 and/or detector 300, on the ceiling, or on the floor. Vision system server 1710 then continually calculates the position of each marker in three dimensions by triangulating the position of the marker from various views generated by cameras 1715a, 1715b, 1715c, . . . 1715n. Vision system server 1710 then uses the locations of the system and subject markers in various vector calculations to derive the position of subject 605 with respect to robotic array 600 at all times during a scan, and the positions of subject 605 and robotic array 600 with respect to a global ground coordinate system to normalize and systemize vectorial calculations. This information is then used by image processing server 195 to adjust for any offsets in frames while processing the multi-dimensional image of subject 605. In alternative embodiments, such as when tomosynthesis scans are performed (which require precise positioning of emitter 200 and detector 300 with respect to subject 605), the location of subject 605 is used to dynamically adjust the trajectories of scanning robots 100a, 100b during a scan to compensate for any motion in subject 605. In other embodiments, the location of subject 605 may also be used to move emitter 200 and/or detector 300 to prevent a collision with subject 605, for example, if subject 605 trips or otherwise moves rapidly in the direction of one of scanning robots 100a, 100b or into the trajectories of robots 100a, 100b.

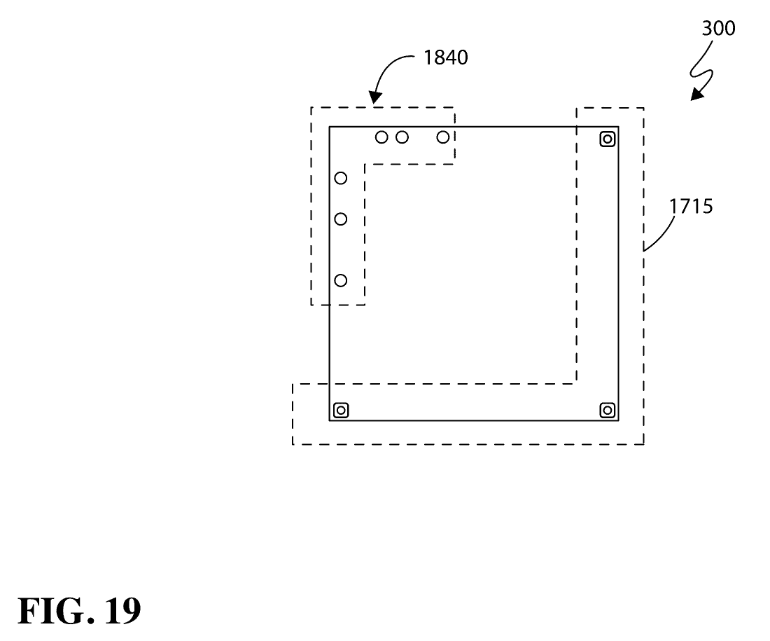

In still another embodiment, the subject and system markers are placed in and around robotic array 600 in accordance with predefined geometric patterns. Vision system server 1710 uses the predefined geometric patterns to better distinguish between the system and subject markers during processing. For example, in one embodiment, as shown in FIG. 19, system markers 1840 are placed in a predefined geometric pattern on detector 300, together with cameras 1715 (as described above, other cameras 1715 may be placed at other locations in and around robotic array 600). Vision system server 1710 then uses triangulation to determine the respective locations of all markers in three dimensions, including system markers 1840 and subject markers (not shown). After the locations of all markers are determined, vision system server 1710 searches for the predefined geometric pattern. Once found, vision system server 1710 assigns the markers forming the geometric pattern to the coordinate system of robotic array 600. It should be appreciated that an alternative geometric pattern of markers may also be positioned on subject 605 to assist vision system server 1710 to identify and distinguish subject markers from system markers. Markers may also be placed in predefined geometric patterns on other features in and around robotic array 600 to assist vision system server 1710 to distinguish between different structures.

### Coordinate Systems and Transformation

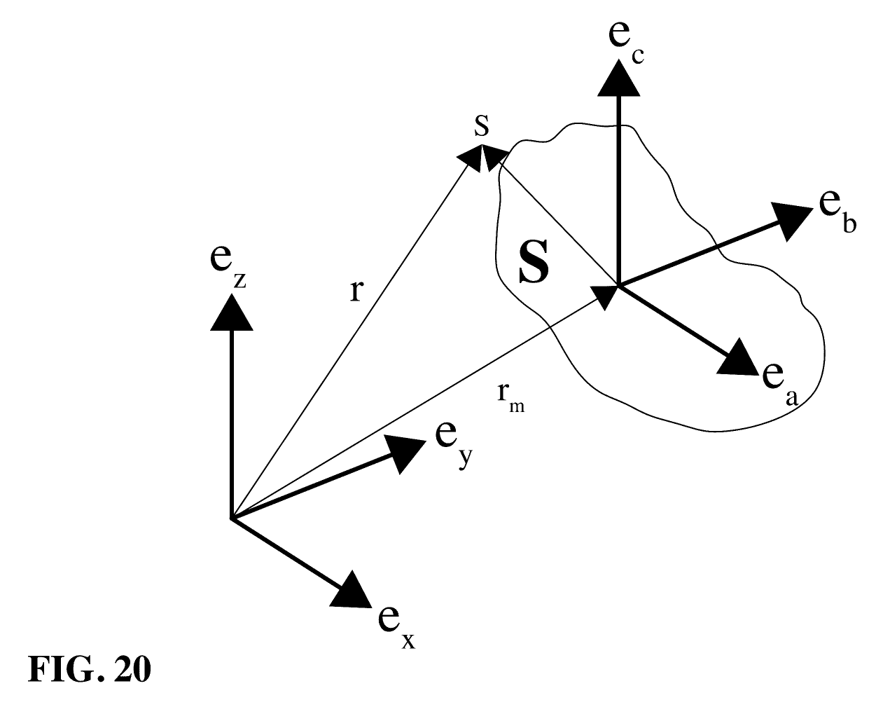

Vision system server 1710 may employ any algorithm for determining the offset of frames in an image set using the subject and system markers, such as those that use the locations of the subject and system markers to determine the relative positions of array 600 and subject 605 coordinate systems with respect to a fixed coordinate system. In one algorithm, for example, the laboratory fixed coordinate system may be designated by xyz and the body reference system by abc. The location of a point S(a/b/c) in the body reference system is defined by the radius vector s=aeₐ+be_{b}+ce_{c}. Consider the reference system to be embedded into the laboratory system. Then the radius vector rm=xₘeₓ+yₘe_{y}+zₘe_{z} describes the origin of the reference system in the laboratory system. The location of S(x/y/z) is now expressed by the coordinates a, b, c. The vector equation r=rₘ+s gives the radius vector for point S in the laboratory system (see FIG. 20). Employing the full notation: r=(xeₓ+ye_{y}+ze_{z})=(xₘeₓ+yₘe_{y}+zₘe_{z})+(aeₐ+beₐ+ce_{c}). A set of transformation equations results after some intermediate matrix algebra to describe the coordinates.

The scalar products of the unit vectors in the xyz and abc systems produce a set of nine coefficients Cᵢⱼ. The cosine of the angle between the coordinate axes of the two systems corresponds to the value of the scalar products. Three “direction cosines” define the orientation of each unit vector in one system with respect to the three unit vectors of the other system. Due to the inherent properties of orthogonality and unit length of the unit vectors, there are six constraints on the nine direction cosines, which leaves only three independent parameters describing the transformation. Employing the matrix notation of the transformation equation, the below is obtained:

$$[ \begin{matrix}x \\ y \\ z\end{matrix} ] = [ \begin{matrix}{x}_{m} \\ {y}_{m} \\ {z}_{m}\end{matrix} ] + [ \begin{matrix}{c}_{11} & {c}_{12} & {c}_{13} \\ {c}_{21} & {c}_{22} & {c}_{23} \\ {c}_{31} & {c}_{32} & {c}_{33}\end{matrix} ] * [ \begin{matrix}a \\ b \\ c\end{matrix} ]$$

In coordinate transformations the objects remain unchanged and only their location and orientation are described in a rotated and possibly translated coordinate system. If a measurement provides the relative spatial location and orientation of two coordinate systems the relative translation of the two systems and the nine coefficients Cᵢⱼ can be calculated. The coefficients are adequate to describe the relative rotation between the two coordinate systems.

### Translation in Three-Dimensional Space

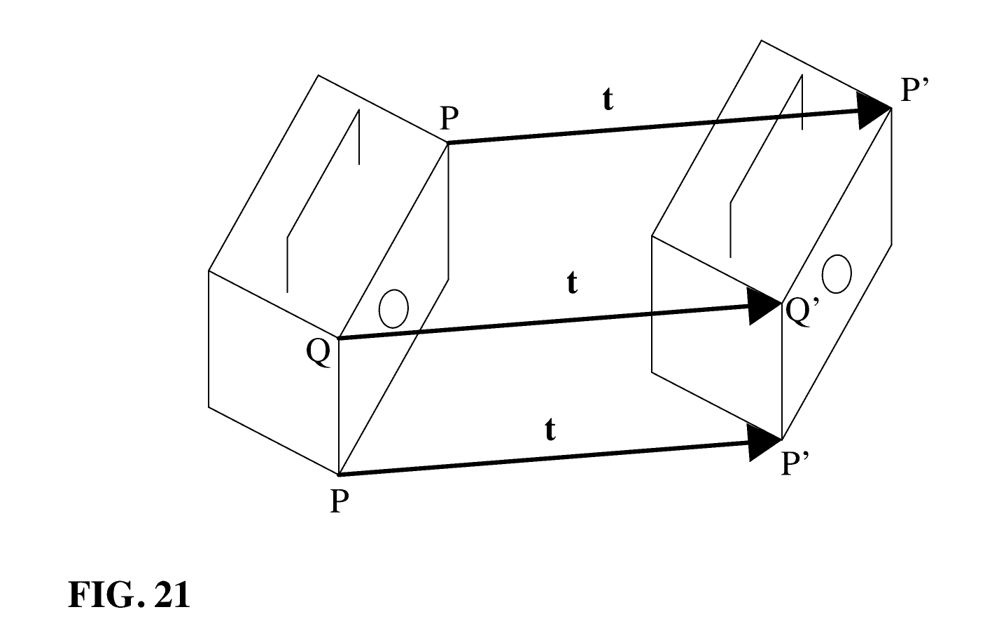

In translation in 3D space the rigid object moves parallel to itself (see FIG. 21). Pure translation in 3D space leaves the orientation of the body unchanged as in the case of pure 2D translation.

### Rotations About the Coordinate Axis

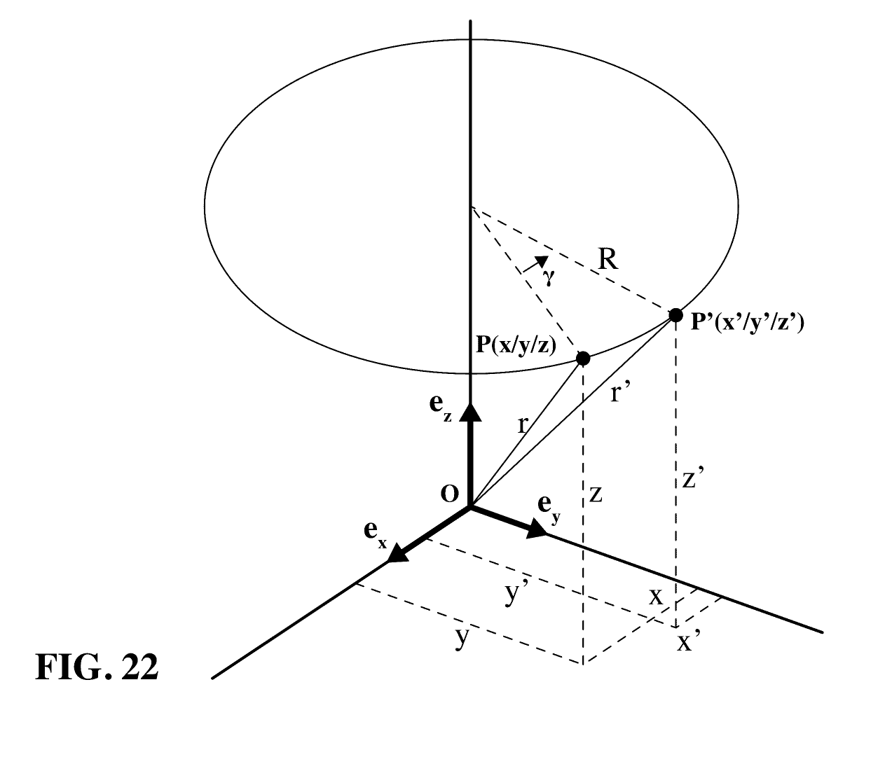

A rotation in three-dimensional space is defined by specifying an axis and an angle of rotation, such as shown in FIG. 22. The axis can be described by its 3D orientation and location. A rotation, as does the translation explained earlier, leaves all the points on the axis unchanged; all other points move along circular arcs in planes oriented perpendicular to the axis.

This rotation moves an arbitrary point P to location P′ with constant distance z from the xy-plane(z =z′). This produces the following matrix notation for the respective equations for the rotation that changes x- and y-coordinates but leaves the z-coordinate unchanged.

$${r}^{′} = [ \begin{matrix}{x}^{′} \\ {y}^{′} \\ {z}^{′}\end{matrix} ] = [ \begin{matrix}cos γ & - sin γ & 0 \\ sin γ & cos γ & 0 \\ 0 & 0 & 1\end{matrix} ] * [ \begin{matrix}x \\ y \\ z\end{matrix} ] = {D}_{z} ( γ ) r$$

The matrix describing a rotation about the z-axis is designated D_{z}(γ). The matrices describing a rotation about the y-axis through angle β and about x-axis through angle α are similar.

$${r}^{′} = [ \begin{matrix}{x}^{′} \\ {y}^{′} \\ {z}^{′}\end{matrix} ] = [ \begin{matrix}cos β & 0 & sin β \\ 0 & 1 & 0 \\ - sin β & 0 & cos β\end{matrix} ] * [ \begin{matrix}x \\ y \\ z\end{matrix} ] = {D}_{y} ( β ) r$$

$${r}^{′} = [ \begin{matrix}{x}^{′} \\ {y}^{′} \\ {z}^{′}\end{matrix} ] = [ \begin{matrix}1 & 0 & 0 \\ 0 & cos α & - sin α \\ 0 & sin α & cos α\end{matrix} ] * [ \begin{matrix}x \\ y \\ z\end{matrix} ] = {D}_{x} ( α ) r$$

### Combined Rotations as a Result of a Sequence of Rotations

Assume that the first rotation of a rigid body occurs about the z-axis of a coordinate system. The rotation matrix related to the unit vectors eₓ, e_{y}, e_{z} is

$${D}_{z} ( γ = 90 ° ) = [ \begin{matrix}0 & - 1 & 0 \\ 1 & 0 & 0 \\ 0 & 0 & 1\end{matrix} ]$$

The second rotation occurs supposedly about the x′-axis, i.e., about a body-fixed axis on the body (previously rotated about its z-axis). The rotation matrix related to the unit vectors e′ₓ, e′_{y}, e′_{z}, is

$${D}_{{x}^{′}} ( α = 90 ° ) = [ \begin{matrix}1 & 0 & 0 \\ 0 & 0 & - 1 \\ 0 & 1 & 0\end{matrix} ]$$

Matrix intermediate calculation here gives:

r^{″}=D_{z}_{^{′}}*Dₓ_{^{′}}*r

In this calculation the sequence of the matrices is very important especially as this sequence differs from what one might expect. First, the matrix of the second partial rotation acts on the vector r and then, in a second step on the matrix of the first partial rotation. If the sequence of the two partial rotations is interchanged, the combined rotation is described by

r^{″}=Dₓ*D_{z}_{^{′}}*r

For rotations about body-fixed axes it is true that in general, the matrix of the last rotation in the sequence of rotations is the first one to be multiplied by the vector to be rotated. The matrix B describing the image resulting from n partial rotations about body-fixed axes is composed according to the formula:

B_{body-fixed}=D₁*D₂*D₃* . . . *Dₙ₋₁

where the indices indicate the sequence of the rotations. Alternatively if the n rotation were to be produced about axes fixed in space (i.e., fixed in the ground, laboratory frame) and not about body-fixed axes, the sequence of the matrices in the matrix product would be different:

B_{space-fixed}=Dₙ*Dₙ₋₁* . . . *D₂*D₁

### Euler and Bryant-Cardan Angles

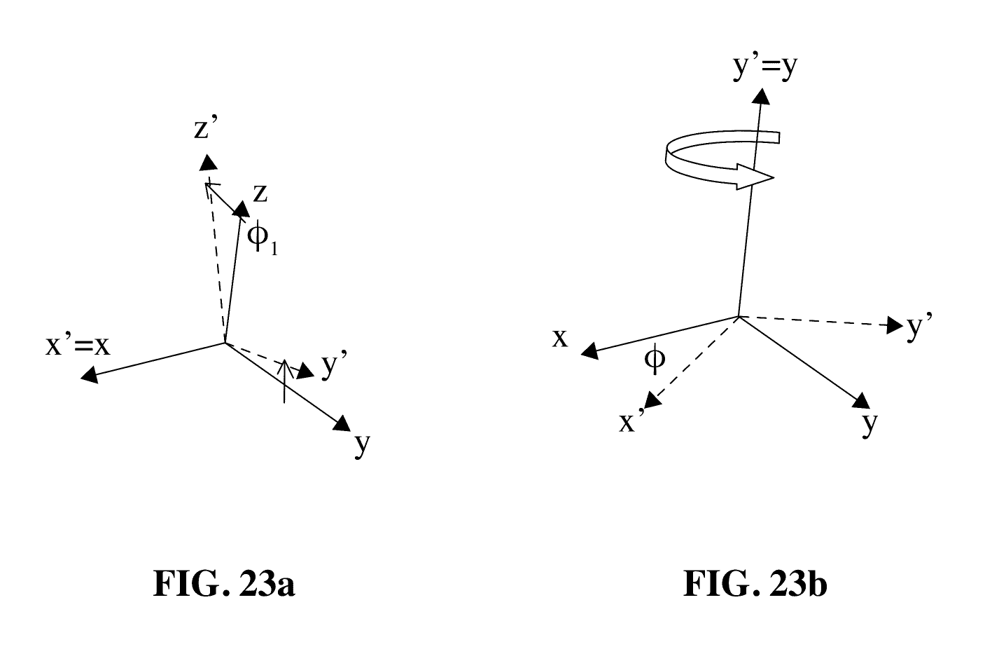

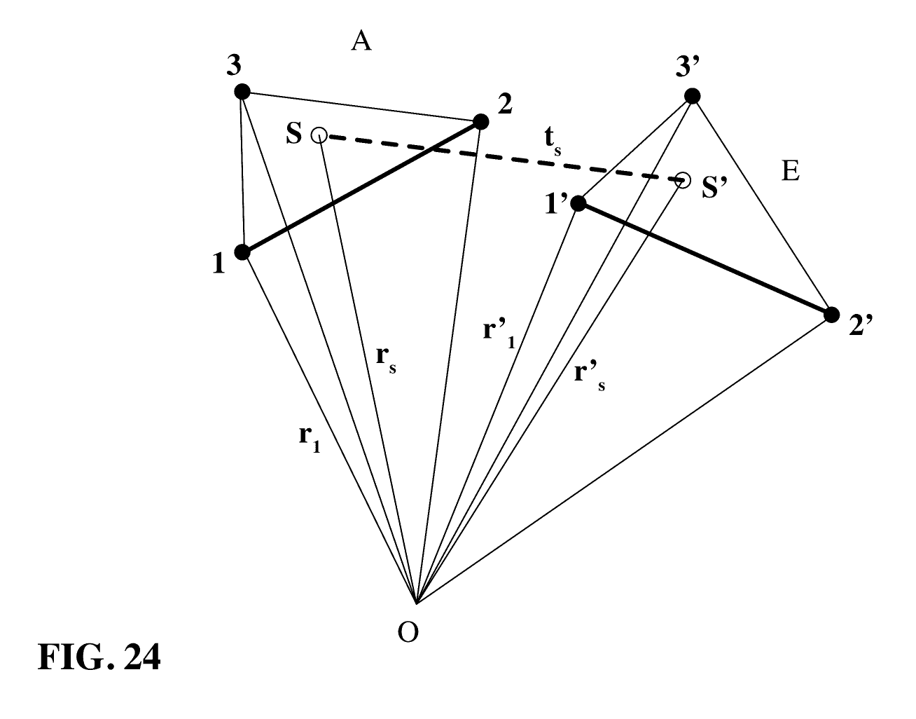

Any desired orientation of a body can be obtained by performing rotations about three axes in sequence. There are, however, many ways of performing three such rotations. One can do this task at random but for reasons of clarity two conventions are frequently used: the Euler's and Bryant-Cardan's rotations. In the Euler notation the general rotation is decomposed of three rotations about body-fixed axes in the following manner: Rotation 1: about a z-axis through the angle θ rotation matrix D_{z}(θ); (see FIGS. 23a and 23b); rotation 2: about the x′-axis through the angle θ rotation matrix D_{x′}(θ); and rotation 3: about the z″-axis through the angle ψ rotation matrix D_{z″}(ψ).

The matrix describing Euler' s combined rotation is given by the matrix product:

B=D_{z}(φ)*Dₓ_{^{′}}(θ)*D_{z}_{^{″}}(Φ) (Euler)

According to the Bryant and Cardan the general rotation is decomposed of three rotations about body-fixed axes in the following manner: rotation 1: about the x-axis through the angle φ₁ rotation matrix Dₓ(φ₁) (see FIGS. 23a and 23b); rotation 2: about the y′-axis through the angle φ₂ rotation matrix D_{y′}(φ₂); rotation 3: about the z″-axis through the angle φ₃ rotation matrix D_{z″}(φ₃); in which case the matrix of combined rotation is given by:

B=Dₓ(φ₁)*D_{y}_{^{′}}(φ₂)*D_{z}_{^{″}}(φ₃) (Bryant-Cardan)

For reasons of simplicity, single or combined rotations about coordinate axes are presented, but more complicated rotational laws can be applied as rotations about arbitrary axes are dealt with. Rotation and translation can also be integrated into one single motion with Chasles Theorem. Chasles Theorem states that “the general motion in three-dimensional space is helical motion,” or “the basic type of motion adapted to describe any change of location and orientation in three-dimensional space is helical motion.” The relevant axis of rotation is designated the “helical axis.” Chasles Theorem is also known as the “helical axis” theorem.

### Parameters of Body Motion in a Ground-Laboratory Coordinate System

Reconstructing the parameters of the motion of a rigid body in a laboratory coordinate system, the coordinates of three reference points fixed on the body but not lying on a straight line have to be known in the initial state D and the final state E (see FIG. 24). To fit the parameters, the following equation is defined:

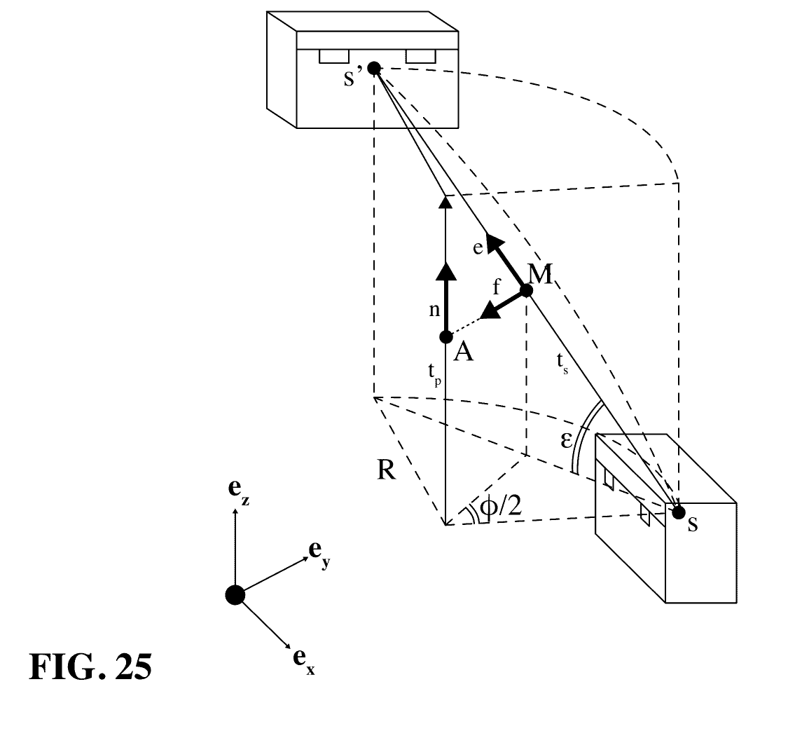

r^{′}=rₛ*D*(r−rₛ)+tₛ

where r refers to the locations of the reference points and the rₛ the location of the geometric center of the reference points in the initial state A. r′ and r′ₛ designate the locations of the reference points and their geometric centers in the final state E. The steps of the calculation are then: (i) calculation of the translation vector tₛ from A to E and reversal of the translation; (ii) determination of the rotation matrix D; (iii) with D and the translation vector already determined in step (i) being known, the Chasles Theorem can be used to interpret the motion as helical motion; (see FIG. 24); and (iv) Euler or Bryant-Cardan interpretations can be used as alternatives to the above (see FIG. 25).

In many cases in motion analysis as it associates with imaging the problem is not in describing the motion of a body in a ground-laboratory coordinate system but in describing the relative motion of two bodies. One example of such relative motion is that of the foot relative to the motion of the shank or the detector with respect to the human head. If one succeeds in fixing a “measurement coordinate system” on one of the bodies, for example on the shank, the problem can be reduced to that discussed above. The motion of the foot would then be observed in the coordinate system of the shank and interpreted according to one of the above conventions (Euler' s angles etc.). Say that the locations of reference points fixed to the shank and to the foot have been acquired simultaneously in a ground-laboratory coordinate system, a number of calculation steps have to be completed before the relative motion between the two segments can be analyzed: (i) from the geometric centers of the reference points on the shank, the translation of the shank is calculated and reversed. A similar procedure is applied to the reference points of the foot; (ii) the rotation matrix that images the already translated reference points of the shank in the final state on to its reference points in the initial state is calculated iteratively. This rotation matrix is then applied to the already translated reference points of the foot. These transformations cause the initial and final state of the reference points of the shank to coincide. Hence the motion of the shank in the laboratory system is described; and (iii) the remaining differences in the locations of the reference points on the foot in the initial and the final state now characterize the relative motion of the foot with respect to the shank. This is similar to analyzing the motion of a single body in the laboratory coordinate system or to analyzing relationships between the robots, the imaging components and the patient segments. The direction and location of the helical axis and the corresponding angle of rotation or, alternatively the translation vector and the sets of angles, according to Euler or to Bryant-Cardan, can be determined.

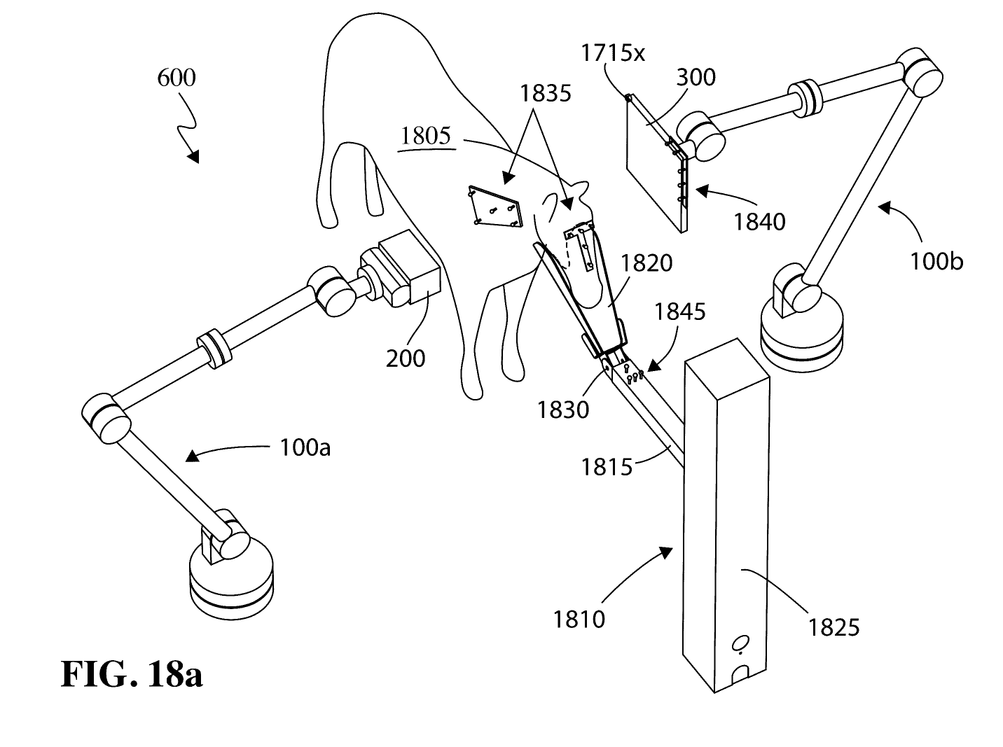

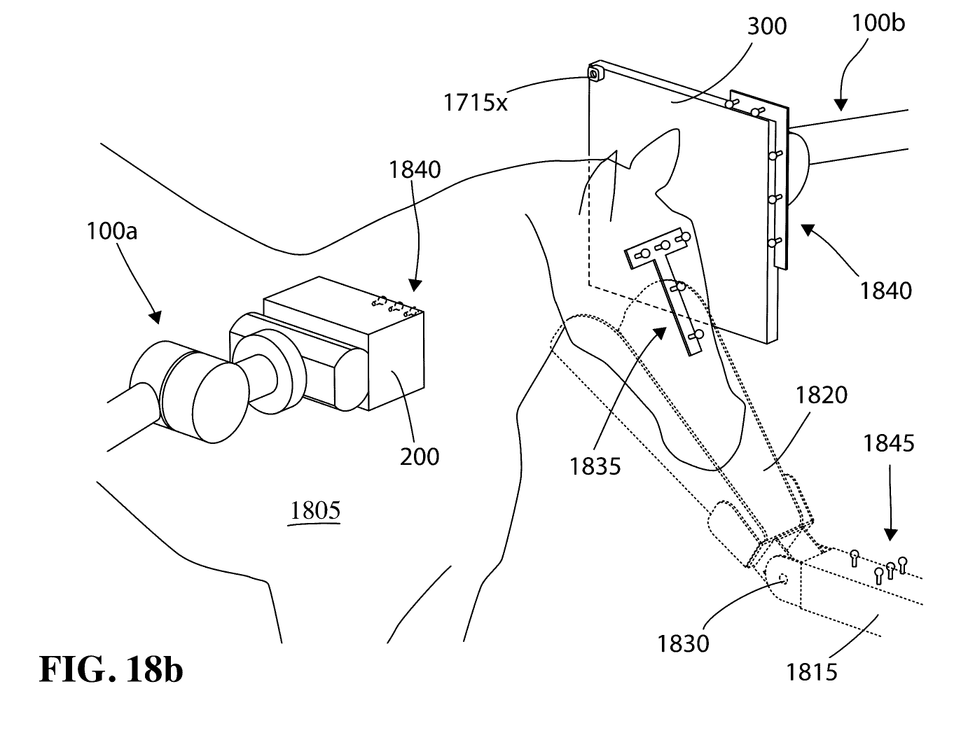

Referring now to FIGS. 18a and 18b, there is seen a robotic array 600 performing a head scan of a horse 1805 in a robotic scanning system 1705 with offset correction capabilities. As shown in FIG. 18, a stand 1810 (which may be rigidly fixed to a floor) is positioned within robotic array 600 to assist in keeping the head of horse 1805 steady during the scan. Stand 1810 includes a base unit 1825 coupled to arm 1815. Arm 1815 is slidably adjustable in the vertical direction with respect to base unit 1825, thereby permitting arm 1815 to adjust to the height of different sized horses. Arm 1815 is also coupled to a cradle 1820 sized and shaped to receive the head of horse 1805. Cradle joint 1830 permits cradle 1820 to be selectively positioned into any of various angular positions with respect to arm 1815. This permits cradle 1820 to comfortably accommodate the shapes and neck-to-head angles of various different sized horse heads.

As shown in FIGS. 18a and 18b, subject markers 1835 are positioned at various locations on horse 1805, such as on the head, torso and legs. System markers 1840 are also placed at various locations within robotic array 600, such as on the emitter 200 and detector 300. It should be appreciated that system markers may be placed at other locations, such as on the floor, on a wall, on arms 130, 145 of robots 100a, 100b or at any other location in and around robotic array 600. In the embodiment shown in FIG. 18, markers 1845 (stand markers) are also placed on stand 1810. Subject markers 1835, system markers 1840 and stand markers 1845 are viewed by cameras 1715a, 1715b, 1715c, . . . 1715n (including camera 1715x attached to detector 300, as shown in FIGS. 18a and 18b) and processed by vision system server 1710 to dynamically determine the respective locations of horse 1805, robotic array 600 and stand 1810 with respect to one another. In one embodiment, this is done by using the locations of markers 1835, 1840, 1845 to dynamically determine the positions of coordinate systems assigned to horse 1805, robotic array 600, and stand 1810, and the relative locations of the origins of these coordinate systems with respect to the origin of another fixed, stationary coordinate system (such as, for example, by employing the algorithms described above). The relative locations of the origins of these coordinate systems are then used by vision system server 1710 to produce one or more correction vectors to correct for any frame offsets caused by movement of horse 1805 and/or stand 1810 with respect to robotic array 600 during the scan. The correction vectors are used by image processing server 195 to correct for frame offsets or, alternatively or in conjunction with such correction, may be used to dynamically adjust the trajectories of emitter 200 and/or detector 300 during the scan. The correction vectors may also be used to move emitter 200 and/or detector 300 to prevent a collision with subject 605, for example, if subject 605 trips or otherwise moves rapidly in the direction of one of scanning robots 100a, 100b or into the trajectories of robots 100a, 100b. in this manner, the correction vectors enable a secondary feature for enhancing patient safety.

While the present invention has been illustrated by description of various embodiments and while those embodiments have been described in considerable detail, it is not the intention of applicant to restrict or in any way limit the scope of the invention to such details. Additional advantages and modifications will readily appear to those skilled in the art. The invention in its broader aspects is therefore not limited to the specific details and illustrative examples shown and described. Accordingly, departures may be made from such details without departing from the spirit or scope of the invention.

What is claimed is:

1. A robotic scanning system, comprising:
a robotic array having at least one set of automated scanning robots configured to perform a radiological scan on a subject;
a control unit in electrical communication with the robotic array, the control unit configured to control the set of scanning robots to perform the radiological scan;
a work station in electrical communication with the control unit, the work station being configured to receive scan settings from a user and to direct the control unit to perform the radiological scan, the work station being configured to direct the control unit to perform any of a plurality of different types of radiological scans selectable by the user; and
an image processing device in electrical communication with the control unit, the image processing device configured to receive scan data from the robotic array and to produce image data indicative of a multi-dimensional image of at least a portion of the subject.

2. The robotic scanning system of claim 1, wherein the set of scanning robots includes first and second scanning robots, each of the first and second scanning robots being attached to a respective radiological unit.

3. The robotic scanning system of claim 2, wherein the radiological unit is constructed as a module configured to be selectively attached to and detached from at least one of the first and second scanning robots.

4. The robotic scanning system of claim 3, wherein at least one of the first and second scanning robots is configured to selectively attach itself to the radiological unit.

5. The robotic scanning system of claim 3, wherein a first radiological unit is attached to the first scanning robot and a second radiological unit is attached to the second scanning robot, the first radiological unit attached to the first scanning robot being an emitter and the second radiological unit attached to the second scanning robot being a detector.

6. The robotic scanning system of claim 1, wherein the plurality of different types of radiological scans include a panoramic scan, a tomosynthesis scan, a volumetric computerized axial tomography scan, a densitometry scan, a biplane dynamic radiographic roentgen stereophotogrammetric scan, and a molecular (gamma) scan.

7. The robotic scanning system of claim 6, wherein the robotic array includes a plurality of sets of automated scanning robots, the plurality of different types of radiological scans including a roentgen stereophotogrammetric panoramic scan, a roentgen stereophotogrammetric tomosynthesis scan, and a biplane dynamic radiographic roentgen stereophotogrammetric scan.

8. The robotic scanning system of claim 7, wherein each set of scanning robots includes first and second scanning robots, each of the first and second scanning robots being attached to a respective radiological unit.

9. The robotic scanning system of claim 8, wherein the radiological unit is constructed as a module configured to be selectively attached to and detached from at least one of the first and second scanning robots.

10. The robotic scanning system of claim 9, wherein at least one of the first and second scanning robots is configured to selectively attach itself to the radiological unit.

11. The robotic scanning system of claim 9, wherein a first radiological unit is attached to the first scanning robot and a second radiological unit is attached to the second scanning robot, the first radiological unit attached to the first scanning robot being an emitter and the second radiological unit attached to the second scanning robot being a detector.

12. The robotic scanning system of claim 2, wherein the radiological unit includes an emitter, the system further comprising:
a high-speed shutter coupled to the emitter and configured to operate synchronously with an x-ray generator to intermittently block emission of a beam emitted from the emitter.

13. The robotic scanning system of claim 1, further comprising:
a vision system device in electrical communication with the control unit; and
a plurality of cameras in electrical communication with the vision system device, the plurality of cameras being configured to view a plurality of markers positioned within an operational envelope of the robotic array, each marker having a respective location within the operational envelope,
wherein the vision system device is configured to generate correction information in accordance with the locations of the plurality of markers within the operational envelope.

14. The robotic scanning system of claim 13, wherein the subject is an animal.

15. The robotic scanning system of claim 13, wherein the subject is an inanimate object.

16. The robotic scanning system of claim 13, wherein the correction information is used to at least one of (i) correct for offsets of frames of an image set and (ii) modify a trajectory of at least one of the scanning robots.

17. The robotic scanning system of claim 16, wherein the correction information is used to modify a trajectory of at least one of the scanning robots to avoid a collision of the scanning robot with the subject or with another object.

18. The robotic scanning system of claim 13, wherein the plurality of markers positioned within the operational envelope include subject markers positioned on the subject and system markers positioned on at least one of the scanning robots of the robotic array.

19. The robotic scanning system of claim 18, wherein at least some of the plurality of markers are positioned within the operational envelope in a predefined geometric pattern to assist the vision system device to distinguish between the subject and system markers.

20. The robotic scanning system of claim 18, wherein the set of scanning robots includes first and second scanning robots, each of the first and second scanning robots being attached to a respective radiological unit, the radiological unit being constructed as a module configured to be selectively attached to and detached from at least one of the first and second scanning robots.

21. The robotic scanning system of claim 20, wherein at least one of the first and second scanning robots is configured to selectively attach itself to the radiological unit.

22. The robotic scanning system of claim 20, wherein the radiological unit attached to the first scanning robot is an emitter and the radiological unit attached to the second scanning robot is a detector.

23. The robotic scanning system of claim 16, wherein the correction information is generated by the vision system device at least in part by (i) determining a position of a first origin of a first coordinate system assigned to the subject, (ii) determining a position of a second origin of a second coordinate system assigned to the robotic array, and (iii) generating at least one correction vector in accordance with the positions of the first and second origins with respect to an origin of a fixed third coordinate system.

24. The robotic scanning system of claim 13, wherein the subject is an animal.

25. The robotic scanning system of claim 13, wherein the subject is an inanimate object.

26. The robotic scanning system of claim 24, wherein the correction information is used by the image processing device to at least one of (i) correct for offsets of frames of an image set and (ii) modify a trajectory of at least one of the scanning robots.

27. The robotic scanning system of claim 26, wherein the animal is a horse having a head, the radiological scan being conducted on the head of the horse, the system further comprising:
a stand having a base unit, an arm coupled to the base unit, and a cradle coupled to the arm and configured to receive the head of the horse during the radiological scan.

28. The robotic scanning system of claim 27, wherein the plurality of markers positioned within the operational envelope include stand markers positioned on the stand.

29. The robotic scanning system of claim 28, wherein at least some of the plurality of markers are positioned within the operational envelope in a predefined geometric pattern to assist the vision system device to distinguish among the subject, system and stand markers.

30. The robotic scanning system of claim 28, wherein the correction information is generated by the vision system device at least in part by (i) determining a position of a first origin of a first coordinate system assigned to the horse, (ii) determining a position of a second origin of a second coordinate system assigned to the robotic array, (iii) determining a position of a third origin of a third coordinate system assigned to the stand, and (iii) generating at least one correction vector in accordance with the positions of the first, second and third origins with respect to an origin of a fixed fourth coordinate system.

31. The robotic scanning system of claim 20, wherein the radiological unit includes an emitter, the system further comprising:
a high-speed shutter coupled to the emitter and configured to operate synchronously with an x-ray generator to intermittently block emission of a beam emitted from the emitter.

32. A robotic scanning system, comprising:
a robotic array having an operational envelope and at least one set of automated scanning robots configured to perform a radiological scan of a subject;
a control unit in electrical communication with the robotic array, the control unit configured to control the set of scanning robots to perform the radiological scan;
a work station in electrical communication with the control unit, the work station being configured to receive scan settings from a user and to direct the control unit to perform the radiological scan;
an image processing device in electrical communication with the control unit, the image processing device configured to receive scan data from the robotic array and to produce image data indicative of a multi-dimensional image of at least a portion of the subject;
a vision system device in electrical communication with the control unit; and
a plurality of cameras in electrical communication with the vision system device, the plurality of cameras being configured to view a plurality of markers positioned within the operational envelope of the robotic array, each marker having a respective location within the operational envelope,
wherein the vision system device is configured to generate correction information in accordance with the locations of the plurality of markers within the operational envelope of the robotic array.

33. The robotic scanning system of claim 32, wherein the correction information is used by the image processing device to at least one of (i) correct for offsets of frames of an image set and (ii) modify a trajectory of at least one of the scanning robots.

34. The robotic scanning system of claim 33, wherein the correction information is used to modify a trajectory of at least one of the scanning robots to avoid a collision of the scanning robot with the subject or with another object.

35. The robotic scanning system of claim 33, wherein the plurality of markers positioned within the operational envelope include subject markers positioned on the subject and system markers positioned on at least one of the scanning robots of the robotic array.

36. The robotic scanning system of claim 35, wherein at least some of the plurality of markers are positioned within the operational envelope in a predefined geometric pattern to assist the vision system device to distinguish between the subject and system markers.

37. The robotic scanning system of claim 35, wherein the set of scanning robots includes first and second scanning robots, each of the first and second scanning robots being attached to a respective radiological unit, the radiological unit being constructed as a module configured to be selectively attached to and detached from at least one of the first and second scanning robots.

38. The robotic scanning system of claim 37, wherein at least one of the first and second scanning robots is configured to selectively attach itself to the radiological unit.

39. The robotic scanning system of claim 33, wherein the correction information is generated by the vision system device at least in part by (i) determining a position of a first origin of a first coordinate system assigned to the subject, (ii) determining a position of a second origin of a second coordinate system assigned to the robotic array, and (iii) generating at least one correction vector in accordance with the positions of the first and second origins with respect to an origin of a fixed third coordinate system.

40. The robotic scanning system of claim 35, wherein the subject is an animal.

41. The robotic scanning system of claim 40, wherein the animal is a horse having a head, the radiological scan being conducted on the head of the horse, the system further comprising:
a stand having a base unit, an arm coupled to the base unit, and a cradle coupled to the arm and configured to receive the head of the horse during the radiological scan,
wherein the plurality of markers positioned within the operational envelope include stand markers positioned on the stand.

42. The robotic scanning system of claim 41, wherein at least some of the plurality of markers are positioned within the operational envelope in a predefined geometric pattern to assist the vision system device to distinguish among the subject, system and stand markers.

43. The robotic scanning system of claim 41, wherein the correction information is generated by the vision system device at least in part by (i) determining a position of a first origin of a first coordinate system assigned to the horse, (ii) determining a position of a second origin of a second coordinate system assigned to the robotic array, (iii) determining a position of a third origin of a third coordinate system assigned to the stand, and (iii) generating at least one correction vector in accordance with the positions of the first, second and third origins with respect to an origin of a fixed fourth coordinate system.

44. A method of conducting a scan on a subject, the method comprising:
placing the subject within an operational envelope of a robotic scanning system, the robotic scanning system including
a robotic array having at least one set of automated scanning robots,
a control unit in electrical communication with the robotic array, the control unit configured to control the set of scanning robots,
a work station in electrical communication with the control unit, the work station being configured to direct the control unit to perform any of a plurality of different types of radiological scans, and
an image processing device in electrical communication with the control unit, the image processing device configured to receive scan data from the robotic array and to produce image data indicative of a multi-dimensional image of at least a portion of the subject;
using the work station of the robotic scanning system to select a type of radiological scan to perform from the plurality of different types of radiological scans;
instructing the work station to perform the selected type of radiological scan on the subject, the robotic array of the scanning system performing the selected radiological scan on the subject; and
viewing a multi-dimensional image of at least a portion of the subject generated by the image processing device.

45. The method of claim 44, wherein the plurality of different types of radiological scans includes a panoramic scan, a tomosynthesis scan, a computerized axial tomography scan, and a bone density scan.

46. The robotic scanning system of claim 45, wherein the robotic array includes a plurality of sets of scanning robots, and the plurality of different types of radiological scans includes a roentgen stereophotogrammetric panoramic scan, a roentgen stereophotogrammetric tomosynthesis scan, and a biplane dynamic radiographic roentgen stereophotogrammetric scan.

47. The robotic scanning system of claim 44, wherein the set of automated scanning robots includes first and second scanning robots, each of the first and second scanning robots being attached to a respective radiological unit.

48. The robotic scanning system of claim 47, wherein the radiological unit is constructed as a module configured to be selectively attached to and detached from at least one of the first and second scanning robots.

49. The robotic scanning system of claim 48, wherein at least one of the first and second scanning robots is configured to selectively attach itself to the radiological unit.

50. The robotic scanning system of claim 47, wherein the radiological unit includes an emitter, the system further comprising:
a high-speed shutter coupled to the emitter and configured to operate synchronously with an x-ray generator to intermittently block emission of a beam emitted from the emitter.
